SEQUENCE LISTING (18 sequences)

SEQ ID NO: 1 - protein, 68 aa, Human
KPVSLSYRCPCRFFESHVARANVKHLKILNTPNCALQIVARLKNNNRQVCIDPKLKWIQEYLEKALNK

SEQ ID NO: 2 - protein, 72 aa, Human
KPVSLSYRCPCRFFESHVARANVKHLKILNTPNCALQIVARLKNNNRQVCIDPKLKWIQEYLEKALNKRFKM

SEQ ID NO: 3 - protein, 98 aa, Human
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 4 - protein, 68 aa, Human
KPVSLSYRCPCRFFESHVARANVAALAILNTPNCALQIVARLKNNNRQVCIDPKLKWIQEYLEKALNK

SEQ ID NO: 5 - protein, 21 aa, Human
MNAKVVVVLVLVLTALCLSDG

SEQ ID NO: 6 - DNA, 267 bp, Human
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaag

SEQ ID NO: 7 - protein, 69 aa, Human
MKPVSLSYRCPCRFFESHVARANVKHLKILNTPNCALQIVARLKNNNRQVCIDPKLKWIQEYLEKALNK

SEQ ID NO: 8 - protein, 65 aa, Human
SLSYRCPCRFFESHVARANVKHLKILNTPNCALQIVARLKNNNRQVCIDPKLKWIQEYLEKALNK

SEQ ID NO: 9 - protein, 66 aa, Human
VSLSYRCPCRFFESHVARANVKHLKILNTPNCALQIVARLKNNNRQVCIDPKLKWIQEYLEKALNK

SEQ ID NO: 10 - protein, 67 aa, Human
MVSLSYRCPCRFFESHVARANVKHLKILNTPNCALQIVARLKNNNRQVCIDPKLKWIQEYLEKALNK

SEQ ID NO: 11 - protein, 69 aa, Human
MKPVSLSYRCPCRFFESHVARANVAALAILNTPNCALQIVARLKNNNRQVCIDPKLKWIQEYLEKALNK

SEQ ID NO: 12 - protein, 68 aa, Human
KPVSLSYRCPCRFFESHVARANVKHLCILNTPNCALQIVARLKNNNRQVCIDPKLKWIQEYLEKALNK

SEQ ID NO: 13 - protein, 300 aa, Human
KPVSLSYRCPCRFFESHVARANVKHLKILNTPNCALQIVARLKNNNRQVCIDPKLKWIQEYLEKALNKEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 119 aa, Human
KPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 15 - protein, 69 aa, Human
KPVSLSYRCPCRFFESHVARANVKHLKILNTPNCALQIVARLKNNNRQVCIDPKLKWIQEYLEKALNNC

SEQ ID NO: 16 - protein, 79 aa, Human
KPVSLSYRCPCRFFESHVARANVKHLKILNTPNCALQIVARLKNNNRQVCIDPKLKWIQEYLEKALNKIWLYGNAETSR

SEQ ID NO: 17 - protein, 65 aa, Human
VSLSYRCPCRFFESHVARANVKHLKILNTPNCALQIVARLKNNNRQVCIDPKLKWIQEYLEKALN

SEQ ID NO: 18 - protein, 66 aa, Human
MVSLSYRCPCRFFESHVARANVKHLKILNTPNCALQIVARLKNNNRQVCIDPKLKWIQEYLEKALN

(19) United States
(12) Patent Application Publication
(10) Pub. No.: US 2008/0253996 A1
(43) Pub. Date: Oct. 16, 2008

(54) Use of Sdf-1 for the Treatment and/or Prevention of Neurological Diseases

(75) Inventors: Ursula Boschert, Troinex (CH); Amanda Proudfoot, Chens Sur Leman (FR); Linda Kadi, Prevessin Moens (FR); Pierre Alain Vitte, Cranves-Sales (FR); Jerome Wojcik, Vesancy (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: LABORATORIES SERONO SA, Coinsins, Vaud (CH)

(21) Appl. No.: 12/067,224

(22) PCT Filed: Oct. 30, 2006
(86) PCT No.: PCT/EP2006/067949

§ 371 Date: Mar. 18, 2008

Related U.S. Application Data
(60) Provisional application No. 60/734,142, filed on Nov. 7, 2005.

(30) Foreign Application Priority Data
Oct. 31, 2005 (EP) 05110206.9

(51) Int. Cl. A61K 38/21; A61K 38/16; A61K 38/14; A61P 25/00

(52) U.S. Cl. 424/85.6; 514/12; 514/8

(57) ABSTRACT

The invention relates to the use of SDF-1, or of an agonist of SDF-1 activity, for the treatment and/or prevention of a neurological disease.

### FIELD OF THE INVENTION

The present invention is generally in the field of neurological diseases associated with neuro-inflammation. More specifically, the present invention relates to the use of SDF-1 for the manufacture of a medicament for treatment and/or prevention of a neurological disease.

### BACKGROUND OF THE INVENTION

Neurological Diseases Associated with Neuro-Inflammation.

Neuro-inflammation is a common feature to most neurological diseases. Many stimuli are triggering neuro-inflammation, which can either be induced by neuronal or oligodendroglial suffering, or be a consequence of a trauma, of a central or peripheral nerve damage or of a viral or bacterial infection. The main consequences of neuro-inflammation are (i) secretion of various inflammatory chemokines by astrocytes, microglia cells; and (ii) recruitment of additional leukocytes, which will further stimulate astrocytes or microglia. In chronic neurodegenerative diseases such as multiple sclerosis (MS), Alzheimer disease (AD) or amyotrophic lateral sclerosis (ALS), the presence of persistent neuro-inflammation is though to participate to the progression of the disease. Neurological diseases associated with neuro-inflammation can also be referred to as neurological inflammatory diseases.

### Chronic Neurodegenerative Diseases

In chronic neurodegenerative diseases, the pathology is associated with an inflammatory response. Recent evidence suggests that systemic inflammation may impact on local inflammation in the diseased brain leading to exaggerated synthesis of inflammatory cytokines and other mediators in the brain, which may in turn influence behavior (Perry, 2004). Chronic neurodegenerative diseases comprise, among others, multiple sclerosis (MS), Alzheimer's disease (AD), Parkinson's disease (PD), Huntington's disease (HD), amyotrophic lateral sclerosis (ALS), multiple system atrophy (MSA), prion disease and Down Syndrome.

Alzheimer's disease (AD) is a disorder involving deterioration in mental functions resulting from changes in brain tissue. This includes shrinking of brain tissues, not caused by disorders of the blood vessels, primary degenerative dementia and diffuse brain atrophy. Alzheimer's disease is also called senile dementia/Alzheimer's type (SDAT). Considerable evidence gained over the past decade has supported the conclusion that neuroinflammation is associated with Alzheimer's disease (AD) pathology (Tuppo and Arias, 2005).

Parkinson's disease (PD) is a disorder of the brain characterized by shaking and difficulty with walking, movement, and coordination. The disease is associated with damage to a part of the brain that controls muscle movement. It is also called paralysis agitans or shaking palsy. Increasing evidence from human and animal studies has suggested that neuroinflammation is an important contributor to the neuronal loss in PD (Gao et al., 2003).

Huntington's Disease (HD) is an inherited, autosomal dominant neurological inflammatory disease. The disease does not usually become clinically apparent until the fifth decade of life, and results in psychiatric disturbance, involuntary movement disorder, and cognitive decline associated with inexorable progression to death, typically 17 years following onset.

Amyptrophic Lateral Sclerosis (ALS) is a disorder causing progressive loss of nervous control of voluntary muscles because of destruction of nerve cells in the brain and spinal cord. Amyotrophic Lateral Sclerosis, also called Lou Gehrig's disease, is a disorder involving loss of the use and control of muscles. The nerves controlling these muscles shrink and disappear, which results in loss of muscle tissue due to the lack of nervous stimulation. Although the root cause of ALS remains unknown, neuroinflammation may play a key role in ALS (Consilvio et al., 2004).

Multiple system atrophy (MSA) is a sporadic, adult-onset neurodegenerative disease of unknown etiology. The condition may be unique among chronic neurodegenerative diseases by the prominent, if not primary, role played by the oligodendroglial cell in the pathogenetic process. Data support a role for inflammation-related genes in risk for MSA (Infante et al., 2005). The major difference to Parkinson's disease is that MSA patients do not respond to L-dopa treatment.

Multiple sclerosis (MS) is an inflammatory demyelinating disease of the central nervous system (CNS) that takes a relapsing-remitting or a progressive course. MS is not the only demyelinating disease. Its counterpart in the peripheral nervous system (PNS) is chronic inflammatory demyelinating polyradiculoneuropathy (CIDP). In addition, there are acute, monophasic disorders, such as the inflammatory demyelinating polyradiculoneuropathy termed Guillain-Barré syndrome (GBS) in the PNS, and acute disseminated encephalomyelitis (ADEM) in the CNS. Both MS and GBS are heterogeneous syndromes. In MS different exogenous assaults together with genetic factors can result in a disease course that finally fulfils the diagnostic criteria. In both diseases, axonal damage can add to a primarily demyelinating lesion and cause permanent neurological deficits. MS is an autoimmune disorder in which leukocytes of the immune system launch an attack on the white matter of the central nervous system (CNS). The grey matter may also be involved. Although the precise etiology of MS is not known, contributing factors may include genetic, bacterial and viral infection. In its classic manifestation (85% of all cases), it is characterized by alternating relapsing/remitting phases, which correspond to episodes of neurological dysfunction lasting several weeks followed by substantial or complete recovery (Noseworthy, 1999). Periods of remission grow shorter over time. Many patients then enter a final disease phase characterized by gradual loss of neurological function with partial or no recovery. This is termed secondary progressive MS. A small proportion (˜15% of all MS patients) suffers a gradual and uninterrupted decline in neurological function following onset of the disease (primary progressive MS).

Prion disease and Down Syndrome have also been shown to involve neuroinflammation (Eikelenboom et al., 2002; Hunter et al., 2004).

### Neurological Inflammatory Diseases Following an Infection

Some neuropathies such as, e.g., acute disseminated encephalomyelitis usually follows a viral infection or viral vaccination (or, very rarely, bacterial vaccination), suggesting an immunologic cause to the disease. Acute inflammatory peripheral neuropathies that follow a viral vaccination or the Guillain-Barré syndrome are similar demyelinating disorders with the same presumed immunopathogenesis, but they affect only peripheral structures.

HTLV-associated myelopathy, a slowly progressive spinal cord disease associated with infection by the human T-cell lymphotrophic virus, is characterized by spastic weakness of both legs.

Central nervous system infections are extremely serious infections; meningitis affects the membranes surrounding the brain and spinal cord; encephalitis affects the brain itself. Viruses that infect the central nervous system (brain and spinal cord) include herpesviruses, arboviruses, coxsackieviruses, echoviruses, and enteroviruses. Some of these infections primarily affect the meninges (the tissues covering the brain) and result in meningitis; others primarily affect the brain and result in encephalitis; many affect both the meninges and brain and result in meningoencephalitis. Meningitis is far more common in children than is encephalitis. Viruses affect the central nervous system in two ways. They directly infect and destroy cells during the acute illness. After recovery from the infection, the body's immune response to the infection sometimes causes secondary damage to the cells around the nerves. This secondary damage (postinfectious encephalomyelitis) results in the child having symptoms several weeks after recovery from the acute illness.

### Neurological Diseases Following Injuries

Injury to CNS induced by acute insults including trauma, hypoxia and ischemia can affect both grey and white matter. Injury to CNS involves neuro-inflammation. For example, leukocyte infiltration in the CNS after trauma or inflammation is triggered in part by up-regulation of the MCP-1 chemokine in astrocytes (Panenka et al., 2001).

Trauma is an injury or damage of the nerve. It may be spinal cord trauma, which is damage to the spinal cord that affects all nervous functions that are controlled at and below the level of the injury, including muscle control and sensation, or brain trauma, such as trauma caused by closed head injury.

Cerebral hypoxia is a lack of oxygen specifically to the cerebral hemispheres, and more typically the term is used to refer to a lack of oxygen to the entire brain. Depending on the severity of the hypoxia, symptoms may range from confusion to irreversible brain damage, coma and death.

Stroke is usually caused by reduced blood flow (ischemia) of the brain. It is also called cerebrovascular disease or accident. It is a group of brain disorders involving loss of brain functions that occurs when the blood supply to any part of the brain is interrupted. The brain requires about 20% of the circulation of blood in the body. The primary blood supply to the brain is through 2 arteries in the neck (the carotid arteries), which then branch off within the brain to multiple arteries that each supply a specific area of the brain. Even a brief interruption to the blood flow can cause decreases in brain function (neurological deficit). The symptoms vary with the area of the brain affected and commonly include such problems as changes in vision, speech changes, decreased movement or sensation in a part of the body, or changes in the level of consciousness. If the blood flow is decreased for longer than a few seconds, brain cells in the area are destroyed (infracted) causing permanent damage to that area of the brain or even death.

Traumatic nerve injury may concern both the CNS or the PNS. Traumatic brain injury, also simply called head injury or closed head injury, refers to an injury where there is damage to the brain because of an external blow to the head. It mostly happens during car or bicycle accidents, but may also occur as the result of near drowning, heart attack, stroke and infections. This type of traumatic brain injury would usually result due to the lack of oxygen or blood supply to the brain, and therefore can be referred to as an “anoxic injury”. Brain injury or closed head injury occurs when there is a blow to the head as in a motor vehicle accident or a fall. There may be a period of unconsciousness immediately following the trauma, which may last minutes, weeks or months. Primary brain damage occurs at the time of injury, mainly at the sites of impact, in particular when a skull fraction is present. Large contusions may be associated with an intracerebral hemorrhage, or accompanied by cortical lacerations. Diffuse axonal injuries occur as a result of shearing and tensile strains of neuronal processes produced by rotational movements of the brain within the skull. There may be small hemorrhagic lesions or diffuse damage to axons, which can only be detected microscopically. Secondary brain damage occurs as a result of complications developing after the moment of injury. They include intracranial hemorrhage, traumatic damage to extracerebral arteries, intracranial herniation, hypoxic brain damage or meningitis.

Spinal cord injuries account for the majority of hospital admissions for paraplegia and tetraplegia. Over 80% occur as a result of road accidents. Two main groups of injury are recognized clinically: open injuries and closed injuries. Open injuries cause direct trauma of the spinal cord and nerve roots. Perforating injuries can cause extensive disruption and hemorrhage. Closed injuries account for most spinal injuries and are usually associated with a fracture/dislocation of the spinal column, which is usually demonstrable radiologically. Damage to the cord depends on the extent of the bony injuries and can be considered in two main stages: primary damage, which are contusions, nerve fibre transections and hemorrhagic necrosis, and secondary damage, which are extradural hematoma, infarction, infection and edema.

Trauma is the most common cause of a localized injury to a single nerve. Violent muscular activity or forcible overextension of a joint may produce a focal neuropathy, as may repeated small traumas (e.g. tight gripping of small tools, excessive vibration from air hammers). Pressure or entrapment paralysis usually affects superficial nerves (ulnar, radial, peroneal) at bony prominences (e.g. during sound sleep or during anesthesia in thin or cachectic persons and often in alcoholics) or at narrow canals (e.g. in carpal tunnel syndrome). Pressure paralysis may also result from tumors, bony hyperostosis, casts, crutches, or prolonged cramped postures (e.g. in gardening). Traumatic injuries can also occur during surgical procedures.

### Peripheral Neuropathy

Peripheral Neuropathy is a syndrome of sensory loss, muscle weakness and atrophy, decreased deep tendon reflexes, and vasomotor symptoms, alone or in any combination. Peripheral Neuropathy is associated with axonal degeneration, a process also referred to as Wallerian degeneration. Neuro-inflammation plays a role in Wallerian degeneration (Stoll et al., 2002).

The disease may affect a single nerve (mononeuropathy), two or more nerves in separate areas (multiple mononeuropathy), or many nerves simultaneously (polyneuropathy). The axon may be primarily affected (e.g. in diabetes mellitus, Lyme disease, uremia or with toxic agents) or the myelin sheath or Schwann cell (e.g. in acute or chronic inflammatory polyneuropathy, leukodystrophies, or Guillain-Barré syndrome). Damage to unmyelinated and myelinated fibers results primarily in loss of temperature and pain sensation; damage to large myelinated fibers results in motor or proprioceptive defects. Some neuropathies (e.g. due to lead toxicity, dapsone use, Lyme disease (caused by tick bite), porphyria, or Guillain-Barré syndrome) primarily affect motor fibers; others (e.g. due to dorsal root ganglionitis of cancer, leprosy, AIDS, diabetes mellitus, or chronic pyridoxine intoxication) primarily affect the dorsal root ganglia or sensory fibers, producing sensory symptoms. Occasionally, cranial nerves are also involved (e.g. in Guillain-Barré syndrome, Lyme disease, diabetes mellitus, and diphtheria). Identifying the modalities involved helps determine the cause.

Multiple mononeuropathy is usually secondary to collagen vascular disorders (e.g. polyarteritis nodosa, SLE, Sjögren's syndrome, RA), sarcoidosis, metabolic diseases (e.g. diabetes, amyloidosis), or infectious diseases (e.g. Lyme disease, HIV infection). Microorganisms may cause multiple mononeuropathy by direct invasion of the nerve (e.g. in leprosy).

Polyneuropathy due to acute febrile diseases may result from a toxin (e.g. in diphtheria) or an autoimmune reaction (e.g. in Guillain-Barré syndrome); the polyneuropathy that sometimes follows immunizations is probably also autoimmune.

Toxic agents generally cause polyneuropathy but sometimes mononeuropathy. They include emetine, hexobarbital, barbital, chlorobutanol, sulfonamides, phenyloin, nitrofurantoin, the vinca alkaloids, heavy metals, carbon monoxide, triorthocresyl phosphate, orthodinitrophenol, many solvents, other industrial poisons, and certain AIDS drugs (e.g. zalcitabine, didanosine).

Chemotherapy-induced neuropathy is a prominent and serious side effect of several commonly used chemotherapy medications, including the Vinca alkaloids (vinblastine, vincristine and vindesine), platinum-containing drugs (cisplatin) and Taxanes (paclitaxel). The induction of peripheral neuropathy is a common factor in limiting therapy with chemotherapeutic drugs.

Nutritional deficiencies and metabolic disorders may result in polyneuropathy. B vitamin deficiency is often the cause (e.g. in alcoholism, beriberi, pernicious anemia, isoniazid-induced pyridoxine deficiency, malabsorption syndromes, and hyperemesis gravidarum). Polyneuropathy also occurs in hypothyroidism, porphyria, sarcoidosis, amyloidosis, and uremia. Diabetes mellitus can cause sensorimotor distal polyneuropathy (most common), multiple mononeuropathy, and focal mononeuropathy (e.g. of the oculomotor or abducens cranial nerves).

Polyneuropathy due to metabolic disorders (e.g. diabetes mellitus) or renal failure develops slowly, often over months or years. It frequently begins with sensory abnormalities in the lower extremities that are often more severe distally than proximally. Peripheral tingling, numbness, burning pain, or deficiencies in joint proprioception and vibratory sensation are often prominent. Pain is often worse at night and may be aggravated by touching the affected area or by temperature changes. In severe cases, there are objective signs of sensory loss, typically with stocking-and-glove distribution. Achilles and other deep tendon reflexes are diminished or absent. Painless ulcers on the digits or Charcot's joints may develop when sensory loss is profound. Sensory or proprioceptive deficits may lead to gait abnormalities. Motor involvement results in distal muscle weakness and atrophy. The autonomic nervous system may be additionally or selectively involved, leading to nocturnal diarrhea, urinary and fecal incontinence, impotence, or postural hypotension. Vasomotor symptoms vary. The skin may be paler and drier than normal, sometimes with dusky discoloration; sweating may be excessive. Trophic changes (smooth and shiny skin, pitted or ridged nails, osteoporosis) are common in severe, prolonged cases.

Nutritional polyneuropathy is common among alcoholics and the malnourished. A primary axonopathy may lead to secondary demyelination and axonal destruction in the longest and largest nerves. Whether the cause is deficiency of thiamine or another vitamin (e.g. pyridoxine, pantothenic acid, folic acid) is unclear. Neuropathy due to pyridoxine deficiency usually occurs only in persons taking isoniazid for tuberculosis; infants who are deficient or dependent on pyridoxine may have convulsions. Wasting and symmetric weakness of the distal extremities is usually insidious but can progress rapidly, sometimes accompanied by sensory loss, paresthesias, and pain. Aching, cramping, coldness, burning, and numbness in the calves and feet may be worsened by touch. Multiple vitamins may be given when etiology is obscure, but they have no proven benefit.

Hereditary neuropathies are classified as sensorimotor neuropathies or sensory neuropathies. Charcot-Marie-Tooth disease is the most common hereditary sensorimotor neuropathy. Less common sensorimotor neuropathies begin at birth and result in greater disability. In sensory neuropathies, which are rare, loss of distal pain and temperature sensation is more prominent than loss of vibratory and position sense. The main problem is pedal mutilation due to pain insensitivity, with frequent infections and osteomyelitis. Hereditary neuropathies also include hypertrophic interstitial neuropathy and Dejerine-Sottas disease.

Malignancy may also cause polyneuropathy via monoclonal gammopathy (multiple myeloma, lymphoma), amyloid invasion, or nutritional deficiencies or as a paraneoplastic syndrome.

While of various etiologies, such as infectious pathogens or autoimmune attacks, neurological inflammatory diseases all cause loss of neurological function and may lead to paralysis and death. Although a few therapeutic agents reducing inflammatory attacks in some neurological inflammatory diseases are available, there is a need to develop novel therapies that could lead to recovery of neurological function.

### SDF-1

Chemokines (chemotactic cytokines) constitute a superfamily of small (8-10 kDa) cytokines that activate seven transmembrane, G protein-coupled receptors that are involved both in basal trafficking and inflammatory responses acting primarily as leukocyte chemoattractants and activators.

Stromal cell-derived factor-1α, SDF-1α, and its 2 isoforms (β,γ) are small chemotactic cytokines that belong to the intercrine family, members of which activate leukocytes and are often induced by proinflammatory stimuli such as lipopolysaccharide, TNF, or IL-1. The intercrines are characterized by the presence of 4 conserved cysteines, which form 2 disulfide bonds. They can be classified into 2 subfamilies. In the CC subfamily, which includes beta chemokine, the cysteine residues are adjacent to each other. In the CXC subfamily, which includes alpha chemokine, they are separated by an intervening amino acid. The SDF-1 proteins belong to the latter group. SDF-1 is a natural ligand of the CXCR4 (LESTR/fusin) chemokine receptor. The alpha, beta and gamma isoforms are a consequence of alternative splicing of a single gene. The alpha form is derived from exons 1-3 while the beta form contains an additional sequence from exon 4. The first three exons of SDF-1γ are identical to those of SDF-1α and SDF-1β. The fourth exon of SDF-1γ is located 3200 bp downstream from the third exon on SDF-1 locus and lies between the third exon and the fourth exon of SDF-1β.

Three new SDF-1 isoforms, SDF-1delta, SDF-1epsilon and SDF-1phi have been described recently (Yu et al., 2006). The SDF-16 isoform is alternatively spliced in the last codon of the SDF-1α open reading frame, resulting in a 731 base-pairs intron, with the terminal exon of SDF-1α being split into two. The first three exons of SDF-1ε and SDF-1Φ are 100% identical to that of SDF-1β and SDF-1γ isoforms.

The SDF-1 gene is expressed ubiquitously with the exception of blood cells it acts on lymphocytes and monocytes but not neutrophils in vitro and is a highly potent chemoattractant for mononuclear cells in vivo. In vitro and in vivo SDF also acts as a chemoattractant for human hematopoietic progenitor cells expressing CD34.

SDF-1 and its receptor, CXCR4, exercise essential functions in the hematopoietic system and the nervous system since deletion of either the ligand or the receptor is embryonic lethal due to abnormal CNS development (Ma et al., 1998; Zou et al., 1998).

SDF-1 α, through interactions with its receptor CXCR4 can directly induce cell death by apoptosis in the human hNT neuronal cell line, which resembles immature post-mitotic cholinergic neurons and has a number of neuronal characteristics (Hesselgesser et al., 1998).

The role of SDF-1 in the developing and mature central nervous system was reviewed by Lazarini et al. (Lazarini et al., 2003).

Chemokines are certainly involved in neuro-inflammation in the CNS, but their activities extend to their role as biologically important peptides directly on neuroepithelial cells (including neurons, astrocytes and oligodendrocytes). In particular, chemokines influence proliferation of oligodendrocyte precursors (OLPs), as illustrated by GRO-α/CXCL1 (Robinson et al., 1998), organization of cerebellar granule cells, in the case of SDF-1α (Zhu et al., 2002) and activation states of microglia as exemplified by fractalkine/CX3CL1 (Zujovic et al., 2000), to name but a few. Thus, in both the immune system and nervous system paradigms, chemokines can perform a wide range of similar activities, including regulation of proliferation, migration, activation and differentiation.

Many chemokines and chemokine receptors are expressed in the CNS, either constitutively or induced by inflammatory mediators. They are involved in many neuropathological processes, including multiple sclerosis (MS) (Bajetto et al., 2001; Sorensen et al., 2002).

The expression of SDF-1 in brain endothelial cells has been shown to favour the recruitment of immune cells to the ischemic CNS (Stumm et al., 2002), suggesting a detrimental role of SDF-1 in neuroinflammation. In the context of aids dementia, SDF-1 was described to induce neurotoxicity by stimulating TNFα production by activated microglia and glutamate release by astrocytes in an gp120 induced in vitro neuroinflammation model (Bezzi et al., 2001; Sorensen et al., 2002). A recent publication described SDF-1α expression in astrocytes of MS lesions (Ambrosini et al., 2005).

Induction of experimental allergic encephalomyelitis (EAE) in the rat was accompanied by increased levels of various chemokine receptors including CXCR4 (Jiang et al., 1998).

In WO00/09152, CXCR4 antagonists have been said to be useful for the treatment of an autoimmune disease, treatment of multiple sclerosis, treatment of cancer and inhibition of angiogenesis.

WO99/50461 discloses methods of treatment of disorders involving aberrant cellular proliferation or deficient cell proliferation by administering compounds that promote or inhibit CXCR4 activity. Inhibitors of the CXCR4 function were claimed for the treatment of cancers and uses of the receptor agonists were claimed for the treatment of disorders in which cell proliferation is deficient or is desired. Disorders in which cell proliferation is deficient include demyelinating lesions of the nervous system in which a portion of the nervous system is destroyed or injured by a demyelinating disease including e.g. multiple sclerosis and lesions of peripheral nervous system.

The therapeutic use of CXCR4/SDF-1 antagonists in neurological diseases has also been suggested. In EP657468B1, the use of SDF-1 is suggested for the treatment of diseases relating to undergrown or abnormal proliferation of hematopoietic cells, neuronal enhancement or depression, prevention or treatment of neuronal injury.

In WO03/062273, an inhibitor of SDF-1 signalling pathway was described for the treatment of inflammation. The therapeutic uses disclosed include inflammation associated with autoimmune diseases or conditions or disorders, where either in the CNS or in any other organ, immune and/or inflammation suppression would be beneficial, chronic neuropathy or Guillain Barre syndrome.

Gleichmann et al. reported a slight transient increase in SDF-1-beta mRNA expression after peripheral nerve lesion. They concluded that their findings demonstrate for the first time a differential expression pattern for SDF-1 isoforms at distinct physiological conditions such as development and injury of the nervous system (Gleichmann et al., 2000).

SDF-1 can interact with Glycosaminoglycans (GAGs), highly variable, branched sugar groups added post-translationally to several proteins, generically called proteoglycans (PGs). Such proteins are present on cell membrane, in the extracellular matrix and in the blood stream, where isolated GAGs can also be present. PGs, or isolated GAGs, can form a complex with soluble molecules, possibly to protect this molecule from proteolysis in the extracellular environment. It has also been proposed that GAGs may help the correct presentation of cell signaling molecules to their specific receptor and, eventually, also the modulation of target cell activation.

In the case of chemokines, the concentration into immobilized gradients at the site of inflammation and, consequently, the interaction with cell receptors and their activation state seem to be modulated by the different forms of GAGs (Hoogewerf et al., 1997). Therefore, it has been suggested that the modulation of such interactions may represent a therapeutic approach in inflammatory disease (Schwarz and Wells, 1999).

A modified SDF-1α, SDF-1 3/6, was generated by combined substitution of the basic cluster of residues Lys24, His25 and Lys27 by Ser (Amara et al., 1999). This mutant was unable to bind heparan sulfate but kept the ability to bind and activate the CXCR4. Another study investigated the effect of single mutations in the same domain and characterized the SDF-1α heparin complex (Sadir et al., 2001). Sadir et al. also suggested the involvement of residues Arg41 and Lys43 in glycosaminoglycan binding.

### SUMMARY OF THE INVENTION

It is the object of the present invention to provide novel means for the treatment and/or prevention of a neurological disease.

In the frame of the present invention, it has been found that administration of SDF-1α, Met-SDF-1α or SDF-1α variant has a beneficial effect in an in vivo animal model of peripheral neurological diseases. SDF-1α and its variant were also shown to inhibit TNF-α and IL-6 in the LPS induced TNF-α release animal model, which is a model of inflammation.

The experimental evidence presented herein therefore provides for a new possibility of treating neurological diseases, in particular those linked to neuronal and glial cell function and neuro-inflammation.

Therefore, the present invention relates to the use of SDF-1 or an agonist of SDF-1 activity, for the manufacture of a medicament for the treatment and/or prevention of a neurological disease.

In accordance with the present invention, SDF-1 may also be used in combination with an interferon or osteopontin or clusterin for treatment and/or prevention of neurological diseases. The use of nucleic acid molecules, expression vectors comprising SDF-1, and of cells expressing SDF-1, for treatment and/or prevention of a neurological disease is also within the present invention.

The invention further provides pharmaceutical compositions comprising SDF-1 and an interferon or osteopontin or clusterin optionally together with one or more pharmaceutically acceptable excipients

### BRIEF DESCRIPTION OF THE DRAWINGS

FIG. 1 shows TNF-α and IL-6 content in pg/ml of mixed cortical cultures pre-incubated at day 14 of cell culture with 0.001, 0.1 and 10 ng/ml of SDF-1α (1.A) or SDF-1α variant (1.B) for three hours at 37° C. then supplemented with 5 ng/ml of LPS for 48 hours. Supernatants were collected at day 16 and the levels of TNF-α and IL-6 were measured via specific ELISAs. As positive controls, cultures were treated with 25 pM of dexamethasone (Dexa), 10 ng/ml of IL-10 or untreated. As negative control, cultures were treated with LPS only.

FIG. 2 shows the mean total number of cells×10⁶±s.e. recruited in the peritoneal cavity at 4 hours after intra peritoneal injection of 200 μl NaCl (0.9%, LPS free; Baseline) or 4 μg of SDF-1α or SDF-1α variant diluted in 200 μl NaCl (0.9%, LPS free).

FIG. 3 shows SDF-1α content in picogram per microgram of total protein (pg/mg) of spinal cord extracts dissected from mice afflicted with EAE at chronic phase compared to untreated mice (control).

FIG. 4 shows the electrophysiological recordings of mice, after a sciatic nerve crush, treated with Vehicle (Saline/0.02% BSA), 3, 10, 30, or 100 μg/kg s.c. of SDF-1α and 30 μg/kg of a reference (positive) control compound (IL-6). Baseline: values registered on the contralateral side of Vehicle treated animals. Recordings were performed at day 7, 15 and 22 post lesion (dpl).

4.A represents the amplitude in millivolt (mV) of the compound muscle action potential.

4.B shows the latency in milliseconds (ms) of the compound muscle action potential.

FIG. 5 shows the electrophysiological recordings of mice, after a sciatic nerve crush, treated with Vehicle (Saline/0.02% BSA) or 30 μg/kg s.c. of SDF-1α variant. Baseline: values registered on the contralateral side of Vehicle treated animals. Recordings were performed at day 7 and 22 post lesion (dpl).

5.A represents the amplitude in millivolt (mV) of the compound muscle action potential.

5.B shows the latency in milliseconds (ms) of the compound muscle action potential.

5.C shows the duration in milliseconds (ms) of the compound muscle action potential.

FIG. 6 shows the electrophysiological recordings of mice, after a sciatic nerve crush, treated with Vehicle (Saline/0.02% BSA) or 100, 30, 10 μg/kg s.c. of Met-SDF-1α. Baseline: values registered on the contralateral side of Vehicle treated animals. Recordings were performed at day 7 and 14 post lesion (dpl).

6.A shows the latency in milliseconds (ms) of the compound muscle action potential.

FIG. 7 shows the results of 100, 30, 10 μg/kg s.c. SDF-1α treatment in the streptozotocin model of diabetic neuropathy (STZ). The positive control molecule is IL-6 at 10 μg/kg s.c.

7.A represents the body weight measurement starting at day 11 to day 40

7.B represents glycemia levels at day 7 post-STZ

7.C shows the latency of the compound muscle action potential measured at day 24 and 40 post STZ

7.D shows the effect of SDF-1α on the sensory nerve conduction velocity

7.E represents the relative myelin thickness at day 40 post STZ with and without SDF-1α treatment expressed as the g-ratio

7.F shows the number of degenerated fibers in the sciatic nerve at day 40 post STZ

7.G represents the density of intra-epidermal nerve fibers at day 40 post STZ

FIG. 8 shows the results of 100, 30, 10 μg/kg s.c. SDF-1α treatment on mechanical and thermal allodynia readouts in the streptozotocin model of diabetic neuropathy (STZ).

8.A represents the threshold pressure measured in the Von Frey Filament Test day 20 post STZ

8.B represents the latency measurement in the 52° C. Hot plate assay day 40 post STZ

FIG. 9 shows the estimated false discovery rate on the Italian primary progressive MS collection plotted against the number of positive markers R for R<100.

FIG. 10 shows the SNP_A-2185631 in the SDF-1 gene.

FIG. 11 shows the predicted amino acid sequences of human SDF-1 splice variants.

FIG. 12 shows that SNP_A-2185631 is in the SDF-1 gene, located in the last intron of SDF-1ε and SDF-1Φ.

### DETAILED DESCRIPTION OF THE INVENTION

In the frame of the present invention, it has been found that administration of SDF-1 has a beneficial effect in an in vivo animal model of peripheral neurological diseases. In a murine model of sciatic nerve crush induced neuropathy, all physiologic parameters relating to nerve regeneration, integrity and vitality were positively influenced by administration of SDF-1α, Met-SDF-1α or SDF-1α variant.

SDF-1α and SDF-1α variant were shown to inhibit TNF-α and IL-6 in the LPS induced TNF-α release animal model, which is a generic model of neuro-inflammation.

A protective effect of SDF-1α in diabetic neuropathy and neuropathic pain is shown in the present invention.

Further, a genetic association between SDF-1 gene and primary progressive MS has been found.

The experimental evidence presented herein therefore provides for a new possibility of treating neurological diseases, in particular those linked to neuronal and glial cell function and neuro-inflammation.

The invention therefore relates to the use of SDF-1 or of an agonist of SDF-1 activity, for the manufacture of a medicament for treatment and/or prevention of a neurological disease.

The term “SDF-1”, as used herein, relates to full-length mature human SDF-1α or a fragment thereof having SDF-1 activity, such as e.g. its binding to the CXCR4 receptor. The amino acid sequence of human SDF-1α is reported herein as SEQ ID NO: 1 of the annexed sequence listing. The term “SDF-1”, as used herein, further relates to any SDF-1 derived from animals, such as murine, bovine, or rat SDF-1, as long as there is sufficient identity in order to maintain SDF-1 activity.

The term “SDF-1”, as used herein, further relates to biologically active muteins and fragments, such as the naturally occurring isoforms of SDF-1. Six alternatively spliced transcript variants of the gene encoding distinct isoforms of SDF-1 have been reported (SDF-1 isoforms α, β, γ, δ, ε and Φ). The sequences of human SDF-1 α, SDF-1β, SDF-1γ, SDF-1-δ, SDF-1ε and SDF-1Φ are reported herein as SEQ ID NO: 1, SEQ ID NO: 2, SEQ ID NO: 3, SEQ ID NO: 14, SEQ ID NO:15 and SEQ ID NO:16, respectively, of the annexed sequence listing.

The term “SDF-1”, as used herein, further encompasses isoforms, muteins, fused proteins, functional derivatives, active fractions, fragments or salts thereof. These isoforms, muteins, fused proteins or functional derivatives, active fractions or fragments retain the biological activity of SDF-1. Preferably, they have a biological activity, which is improved as compared to wild type SDF-1.

The term “SDF-1” in particular includes the human mature isoform SDF-1α identified by SEQ ID NO:1, human mature SDF-13 identified by SEQ ID NO:2, human mature SDF-1γ identified by SEQ ID NO:3, human mature SDF-1-δ identified by SEQ ID NO:14, human mature SDF-1ε identified by SEQ ID NO:15 and human mature SDF-1Φ identified by SEQ ID NO:16; the human mature isoform SDF-1α having an additional N-terminal Methionine and being identified by SEQ ID NO: 7; truncated forms of SDF-1α such as the one corresponding to amino acid residues 4-68 of mature human SDF-1α and being identified by SEQ ID NO:8, the one corresponding to amino acid residues 3-68 of mature human SDF-1α and being identified by SEQ ID NO:9, and the one corresponding to amino acid residues 3-68 of mature human SDF-1α having an additional N-terminal Methionine and being identified by SEQ ID NO:10. Also encompassed by the term SDF-1 are fusion proteins comprising an SDF-1 polypeptide as defined above operably linked to a heterologous domain, e.g., one or more amino acid sequences which may be chosen amongst the following: an extracellular domain of a membrane-bound protein, immunoglobulin constant regions (Fc region), multimerization domains, export signals, and tag sequences (such as the ones helping the purification by affinity: HA tag, Histidine tag, GST, FLAAG peptides, or MBP. Preferred are Fc-fusion proteins of SDF-1α as defined by SEQ ID NO: 13.

The term “SDF-1α variant”, as used herein, relates to a mutant of SDF-1 having a reduced GAG-binding activity. The wording “a reduced GAG-binding activity” or “GAG-binding defective” means that the CC-chemokine mutants have a lower ability to bind to GAGs, i.e. a lower percentage of each of these mutants bind to GAGs (like heparin sulphate) with respect to the corresponding wild-type molecule, as measured with the assays in the following cited prior art disclosing such mutants. In particular, such mutant is the one already disclosed in the prior art with the substitutions Lys24 His25 and Lys27 by Ser (Amara et al J Biol Chem. 1999 Aug. 20; 274(34):23916-25) or by Ala (SEQ ID NO: 4). Other GAG binding defective mutants can be generated by combined substitution of the basic cluster of residues Lys24, His25 and Lys27 and any other residues involved in glycosaminoglycan binding e.g. Arg41 and Lys43 with Ser and/or Ala. Possible combinations can be e.g. Lys24 Lys27, Lys24 His25, His25 Lys27, Lys24 Arg 41, His25 Arg41, Lys27 Arg41, Lys24 Lys43, His25 Lys43, Lys27 Lys43, and Arg41 Lys43.

The term “SDF-1α variant” in particular encompasses the mutant of SDF-1α having reduced GAG binding activity and being identified by SEQ ID NO: 4 (triple mutant of SDF-1α having Lys24Ala, His25Ala, Lys27Ala); the mutant of SDF-1α having an additional initial Methionine residue and having the triple mutation Lys25Ala, His26Ala, Lys28Ala, as identified by SEQ ID NO: 11; and the mutant of SDF-1α of reduced GAG binding activity having a single mutation Lys27Cys and being identified by SEQ ID NO: 12. The SDF-1α variants as herein defined, and in particular the SDF-1α variant identified by SEQ ID NO: 12 can be modified with PEG (poly ethylene glycol), a process known as “PEGylation.” PEGylation can be carried out by any of the PEGylation reactions known in the art (see, for example, EP 0 154 316).

SDF-1 and SDF-1α variants as defined herein and having a deletion of the C-terminal amino acid are also included in the invention.

Particularly preferred forms of SDF-1 having a deletion of the C-terminal amino acid are truncated forms of SDF-1α such as the one corresponding to amino acid residues 3-67 of mature human SDF-1α and being identified by SEQ ID NO:17, and the one corresponding to amino acid residues 3-67 of mature human SDF-1α having an additional N-terminal Methionine and being identified by SEQ ID NO:18

The term “agonist of SDF-1 activity”, as used herein, relates to a molecule stimulating or imitating SDF-1 activity, such as agonistic antibodies of the SDF-1 receptor, or small molecular weight agonists activating signalling through an SDF-1 receptor, e.g. the CXCR4 receptor.

The term “agonist of SDF-1 activity”, as used herein, also refers to agents enhancing SDF-1 mediated activities, such as promotion of cell attachment to extracellular matrix components, morphogenesis of cells of the oligodendrocyte lineage into myelin producing cells, promotion of the recruitment, proliferation, differentiation or maturation of cells of the oligodendrocyte lineage (such as progenitors or precursor cells), or promotion of the protection of cells of the oligodendrocyte lineage from apoptosis and cell injury. Similar activities of SDF-1 also apply to Schwann cells.

In a preferred embodiment of the invention, SDF-1 is SDF-1α.

In a further preferred embodiment of the invention, SDF-1 is SDF-1α variant.

The terms “treating” and “preventing”, as used herein, should be understood as preventing, inhibiting, attenuating, ameliorating or reversing one or more symptoms or cause(s) of neurological disease, as well as symptoms, diseases or complications accompanying neurological disease. When “treating” neurological disease, the substances according to the invention are given after onset of the disease, “prevention” relates to administration of the substances before signs of disease can be noted in the patient.

The term “neurological diseases”, as used herein encompasses all known neurological diseases or disorders, or injuries of the CNS or PNS, including those described in detail in the “Background of the invention”.

Neurological diseases comprise disorders linked to dysfunction of the CNS or PNS, such as diseases related to neurotransmission, headache, trauma of the head, CNS infections, neuro-opthalmologic and cranial nerve disorders, function and dysfunction of the cerebral lobes disorders of movement, stupor and coma, demyelinating diseases, delirium and dementia, craniocervical junction abnormalities, seizure disorders, spinal cord disorders, sleep disorders, disorders of the peripheral nervous system, cerebrovascular disease, or muscular disorders. For definitions of these disorders, see e.g. The Merck Manual for Diagnosis and Therapy, Seventeenth Edition, published by Merck Research Laboratories, 1999.

Neuro-inflammation occurs in distinct neurological diseases. Many stimuli are triggering neuro-inflammation, which can either be induced by neuronal or oligodendroglial suffering, or be a consequence of a trauma, of a central or peripheral nerve damage or of a viral or bacterial infection. The main consequences of neuro-inflammation are (i) secretion of various inflammatory chemokines by astrocytes, microglia cells; and (ii) recruitment of additional leukocytes, which will further stimulate astrocytes or microglia. In chronic neurodegenerative diseases such as multiple sclerosis (MS), Alzheimer disease (AD) or amyotrophic lateral sclerosis (ALS), the presence of persistent neuro-inflammation is though to participate to the progression of the disease. Neurological diseases associated with neuro-inflammation can also be referred to as neurological inflammatory diseases.

In a preferred embodiment of the invention, the neurological disease is associated with inflammation, in particular neuro-inflammation.

Preferably, the neurological diseases of the invention are selected from the group consisting of traumatic nerve injury, stroke, demyelinating diseases of the CNS or PNS, neuropathies and neurodegenerative diseases.

Traumatic nerve injury may concern the PNS or the CNS, it may be brain or spinal cord trauma, including paraplegia, as described in the “background of the invention” above.

In preferred embodiments of the invention, the traumatic nerve injury comprises trauma of a peripheral nerve or trauma of the spinal cord.

Stroke may be caused by hypoxia or by ischemia of the brain. It is also called cerebrovascular disease or accident. Stroke may involve loss of brain functions (neurological deficits) caused by a loss of blood circulation to areas of the brain. Loss of blood circulation may be due to blood clots that form in the brain (thrombus), or pieces of atherosclerotic plaque or other material that travel to the brain from another location (emboli). Bleeding (hemorrhage) within the brain may cause symptoms that mimic stroke. The most common cause of a stroke is stroke secondary to atherosclerosis (cerebral thrombosis), and therefore the invention also relates to the treatment of atherosclerosis.

Peripheral Neuropathy may be related to a syndrome of sensory loss, muscle weakness and atrophy, decreased deep tendon reflexes, and vasomotor symptoms, alone or in any combination. Neuropathy may affect a single nerve (mononeuropathy), two or more nerves in separate areas (multiple mononeuropathy), or many nerves simultaneously (polyneuropathy). The axon may be primarily affected (e.g. in diabetes mellitus, Lyme disease, or uremia or with toxic agents), or the myelin sheath or Schwann cell (e.g. in acute or chronic inflammatory polyneuropathy, leukodystrophies, or Guillain-Barré syndrome). Further neuropathies, which may be treated in accordance with the present invention, may e.g. be due to lead toxicity, dapsone use, tick bite, porphyria, or Guillain-Barré syndrome, and they may primarily affect motor fibers. Others, such as those due to dorsal root ganglionitis of cancer, leprosy, AIDS, diabetes mellitus, or chronic pyridoxine intoxication, may primarily affect the dorsal root ganglia or sensory fibers, producing sensory symptoms. Cranial nerves may also be involved, such as e.g. in Guillain-Barré syndrome, Lyme disease, diabetes mellitus, and diphtheria.

Alzheimer's disease is a disorder involving deterioration in mental functions resulting from changes in brain tissue. This may include shrinking of brain tissues, primary degenerative dementia and diffuse brain atrophy. Alzheimer's disease is also called senile dementia/Alzheimer's type (SDAT).

Parkinsons's disease is a disorder of the brain including shaking and difficulty with walking, movement, and coordination. The disease is associated with damage to a part of the brain that controls muscle movement, and it is also called paralysis agitans or shaking palsy.

Huntington's Disease is an inherited, autosomal dominant neurological disease. The genetic abnormality consists in an excess number of tandemly repeated CAG nucleotide sequences. Other diseases with CAG repeats include, for example, spinal muscular atrophies (SMA), such as Kennedy's disease, and most of the autosomal dominant cerebellar ataxias (ADCAs) that are known as spinocerebellar ataxias (SCAs) in genetic nomenclature.

Amyptrophic Lateral Sclerosis, ALS, is a disorder causing progressive loss of nervous control of voluntary muscles, including of destruction of nerve cells in the brain and spinal cord. Amyotrophic Lateral Sclerosis, also called Lou Gehrig's disease, is a disorder involving loss of the use and control of muscles.

Multiple Sclerosis (MS) is an inflammatory demyelinating disease of the central nervous system (CNS) that takes a relapsing-remitting or a progressive course. MS is not the only demyelinating disease. Its counterpart in the peripheral nervous system (PNS) is chronic inflammatory demyelinating polyradiculoneuropathy (CIDP). In addition, there are acute, monophasic disorders, such as the inflammatory demyelinating polyradiculoneuropathy termed Guillain-Barré syndrome (GBS) in the PNS, and acute disseminated encephalomyelitis (ADEM) in the CNS. Further neurological disorders comprise neuropathies with abnormal myelination, such as the ones listed in the “Background of the invention” above, as well as carpal tunnel syndrome. Traumatic nerve injury may be accompanied by spinal column orthopedic complications, and those are also within the diseases in accordance with the present invention.

Less well-known neurological diseases are also within the scope of the present invention, such as neurofibromatosis, or Multiple System Atrophy (MSA). Further disorders that may be treated in accordance with the present invention have been described in detail in the “Background of the invention” above.

In a further preferred embodiment, the neurological disease is a peripheral neuropathy, most preferably diabetic neuropathy. Chemotherapy associated/induced neuropathies are also preferred in accordance with the present invention.

The term “diabetic neuropathy” relates to any form of diabetic neuropathy, or to one or more symptom(s) or disorder(s) accompanying or caused by diabetic neuropathy, or complications of diabetes affecting nerves as described in detail in the “Background of the invention” above. Diabetic neuropathy may be a polyneuropathy. In diabetic polyneuropathy, many nerves are simultaneously affected. The diabetic neuropathy may also be a mononeuropathy. In focal mononeuropathy, for instance, the disease affects a single nerve, such as the oculomotor or abducens cranial nerve. It may also be multiple mononeuropathy when two or more nerves are affected in separate areas.

In a further preferred embodiment, the neurological disorder is a demyelinating disease. Demyelinating diseases preferably comprise demyelinating conditions of the CNS, like acute disseminated encephalomyelitis (ADEM) and multiple sclerosis (MS), as well as demyelinating diseases of the peripheral nervous system (PNS). The latter comprise diseases such as chronic inflammatory demyelinating polyradiculoneuropathy (CIDP and acute, monophasic disorders, such as the inflammatory demyelinating polyradiculoneuropathy termed Guillain-Barré syndrome (GBS).

In a further preferred embodiment, the demyelinating disease is multiple sclerosis.

In a particularly preferred embodiment of the invention, the demyelinating disease is primary progressive multiple sclerosis.

In another particularly preferred embodiment of the invention, the demyelinating disease is secondary progressive multiple sclerosis. In yet a further preferred embodiment, the demyelinating disease is selected from chronic inflammatory multiple sclerosis, demyelinating polyneuropathy (CIDP) and Guillain-Barré syndrome (GBS),

A further preferred embodiment of the invention relates to the treatment and/or prevention of a neurodegenerative disease. The neurodegenerative disease is selected from the group consisting of Alzheimer's disease, Parkinson's disease, Huntington's disease and ALS.

Preferably, the SDF-1 is selected from a peptide, a polypeptide or a protein selected from the group consisting of:
(a) polypeptide comprising amino acids of SEQ ID NO: 1
(b) a polypeptide comprising amino acids of SEQ ID NO: 4
(c) a polypeptide comprising amino acids of SEQ ID NO: 7
(d) a polypeptide of (a) to (c) further comprising a signal sequence, preferably amino acids of SEQ ID NO: 5
(e) a mutein of any of (a) to (d), wherein the amino acid sequence has at least 40% or 50% or 60% or 70% or 80% or 90% identity to at least one of the sequences in (a) to (d);
(f) a mutein of any of (a) to (d) which is encoded by a DNA sequence which hybridizes to the complement of the native DNA sequence encoding any of (a) to (d) under highly stringent conditions;
(g) a mutein of any of (a) to (d) wherein any changes in the amino acid sequence are conservative amino acid substitutions to the amino acid sequences in (a) to (d);
(h) a salt or an isoform, fused protein, functional derivative, or active fraction of any of (a) to (d).

Active fractions or fragments may comprise any portion or domain of any of the SDF-1 isoforms, such as an N-terminal portion of a C-terminal portion, or any of SDF-1 isoforms.

The person skilled in the art will appreciate that even smaller portions of SDF-1 may be enough to exert its function, such as an active peptide comprising the essential amino acid residues required for SDF-1 function, such as e.g. its binding to the CXCR4 receptor. Receptor binding can for example be measured by exposing the immobilized receptor to its labelled ligand and unlabeled test protein, whereby a reduction in labelled ligand binding compared to a control is indicative of receptor-binding activity in the test protein. In another assay, the Surface Plasmon Resonance Spectroscopy, the receptor or protein to be analysed is immobilized on a flat sensor ship in a flow chamber, after which a solution containing a prospective interacting partner is passed over the first protein in a continuous flow, Light is directed at a defined angle across the chip and the resonance angle of reflected light is measured; the establishment of a protein-protein interaction causes a change in the angle (e.g. BIACore®, Biacore International AB). Other techniques suitable to analyse protein-protein interactions (e.g. affinity chromatography, affinity blotting and coimmunoprecipitation) or to evaluate binding affinities (e.g. protein affinity chromatography, sedimentation, gel filtration, fluorescence methods, solid-phase sampling of equilibrium solutions, and surface plasmon resonance) have been reviewed by Phizicky E M and Fields S. (Phizicky and Fields, 1995; Sadir et al., 2001).

The person skilled in the art will further appreciate that muteins, salts, isoforms, fused proteins, functional derivatives or active fractions of SDF-1, will retain a similar, or even better, biological activity of SDF-1. The biological activity of SDF-1 and muteins, isoforms, fused proteins or functional derivatives, active fractions or fragments or salts thereof, may be measured in bioassay, using a cellular system.

Preferred active fractions have an activity which is equal or better than the activity of full-length SDF-1, or which have further advantages, such as a better stability or a lower toxicity or immunogenicity, or they are easier to produce in large quantities, or easier to purify. The person skilled in the art will appreciate that muteins, active fragments and functional derivatives can be generated by cloning the corresponding cDNA in appropriate plasmids and testing them in the cellular assay, as mentioned above.

The proteins according to the present invention may be glycosylated or non-glycosylated, they may be derived from natural sources, such as body fluids, or they may preferably be produced recombinantly. Recombinant expression may be carried out in prokaryotic expression systems such as E. coli, or in eukaryotic, such as insect cells, and preferably in mammalian expression systems, such as CHO-cells or HEK-cells. Furthermore, the proteins of the invention can be modified, extended or shortened, by removing or adding N-terminally a Methionine (Met) or aminooxypentane (AOP), as long as the neuroprotective effects are preserved.

As used herein the term “muteins” refers to analogs of SDF-1, in which one or more of the amino acid residues of a natural SDF-1 are replaced by different amino acid residues, or are deleted, or one or more amino acid residues are added to the natural sequence of SDF-1, without changing considerably the activity of the resulting products as compared with the wild-type SDF-1. These muteins are prepared by known synthesis and/or by site-directed mutagenesis techniques, or any other known technique suitable therefore.

Muteins of SDF-1, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Muteins in accordance with the present invention include proteins encoded by a nucleic acid, such as DNA or RNA, which hybridizes to DNA or RNA, which encodes SDF-1, in accordance with the present invention, under moderately or highly stringent conditions. The cDNA encoding SDF-1α is disclosed as SEQ ID NO 6. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

In a preferred embodiment, any such mutein has at least 40% identity or homology with the sequences of SEQ ID NO: 1 to 4 of the annexed sequence listing. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (Smith and Waterman, 1981) and finds the best single region of similarity between two sequences. Other programs for determining identity and/or similarity between sequences are also known in the art, for instance the BLAST family of programs (Altschul et al., 1990; Altschul et al., 1997), accessible through the home page of the NCBI at www.ncbi.nlm.nih.gov) and FASTA (Pearson, 1990; Pearson and Lipman, 1988).

Preferred changes for muteins in accordance with the present invention are what are known as “conservative” substitutions. Conservative amino acid substitutions of SDF-1 polypeptides, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of SDF-1, polypeptides or proteins, for use in the present invention include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al).

The term “fused protein” refers to a polypeptide comprising SDF-1, or a mutein or fragment thereof, fused with another protein, which e.g. has an extended residence time in body fluids. An SDF-1 may thus be fused to another protein, polypeptide or the like, e.g. an immunoglobulin or a fragment thereof.

“Functional derivatives” as used herein, cover derivatives of SDF-1, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of SDF-1, and do not confer toxic properties on compositions containing it.

These derivatives may, for example, include polyethylene glycol side-chains, which may mask antigenic sites and extend the residence of an SDF-1 in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g alkanoyl or carbocyclic aroyl groups) or O-acyl derivatives of free hydroxyl groups (for example that of seryl or threonyl residues) formed with acyl moieties.

As “active fractions” of SDF-1, muteins and fused proteins, the present invention covers any fragment or precursors of the polypeptide chain of the protein molecule alone or together with associated molecules or residues linked thereto, e.g. sugar or phosphate residues, or aggregates of the protein molecule or the sugar residues by themselves, provided said fraction has substantially similar activity to SDF-1.

The term “salts” herein refers to both salts of carboxyl groups and to acid addition salts of amino groups of SDF-1 molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of SDF-1 relevant to the present invention, i.e., neuroprotective effect in a neurological disease.

In a preferred embodiment of the invention, SDF-1 is fused to a carrier molecule, a peptide or a protein that promotes the crossing of the blood brain barrier (“BBB”). This serves for proper targeting of the molecule to the site of action in those cases, in which the CNS is involved in the disease. Modalities for drug delivery through the BBB entail disruption of the BBB, either by osmotic means or biochemically by the use of vasoactive substances such as bradykinin. Other strategies to go through the BBB may entail the use of endogenous transport systems, including carrier-mediated transporters such as glucose and amino acid carriers; receptor-mediated transcytosis for insulin or transferrin; and active efflux transporters such as p-glycoprotein; Penetration, a 16-mer peptide (pAntp) derived from the third helix domain of Antennapedia homeoprotein, and its derivatives. Strategies for drug delivery behind the BBB further include intracerebral implantation.

Functional derivatives of SDF-1 may be conjugated to polymers in order to improve the properties of the protein, such as the stability, half-life, bioavailability, tolerance by the human body, or immunogenicity. To achieve this goal, SDF-1 may be linked e.g. to Polyethlyenglycol (PEG). PEGylation may be carried out by known methods, described in WO 92/13095. For example, SDF-1α could be pegylated at the residues involved in glycosaminoglycan binding e.g. Lys24, His25, Lys27, Arg41 or Lys43.

Therefore, in a preferred embodiment of the present invention, SDF-1 is PEGylated.

In a further preferred embodiment of the invention, the fused protein comprises an immunoglobulin (Ig) fusion. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between SDF-1 sequence and the immunoglobulin sequence, for instance. The resulting fusion protein has improved properties, such as an extended residence time in body fluids (half-life), or an increased specific activity, increased expression level. The Ig fusion may also facilitate purification of the fused protein.

In a yet another preferred embodiment, SDF-1 is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM, for example. Fusion proteins may be monomeric or multimeric, hetero- or homomultimeric. The immunoglobulin portion of the fused protein may be further modified in a way as to not activate complement binding or the complement cascade or bind to Fc-receptors.

Further fusion proteins of SDF-1 may be prepared by fusing domains isolated from other proteins allowing the formation or dimers, trimers, etc. Examples for protein sequences allowing the multimerization of the polypeptides of the Invention are domains isolated from proteins such as hCG (WO 97/30161), collagen X (WO 04/33486), C4BP (WO 04/20639), Erb proteins (WO 98/02540), or coiled coil peptides (WO 01/00814).

The invention further relates to the use of a combination of SDF-1 and an immunosuppressive agent for the manufacture of a medicament for treatment and/or prevention of neurological disorders, for simultaneous, sequential or separate use. Immunosuppressive agents may be steroids, methotrexate, cyclophosphamide, anti-leukocyte antibodies (such as CAMPATH-1), and the like.

The invention further relates to the use of a combination of SDF-1 and an interferon and/or osteopontin and/or clusterin, for the manufacture of a medicament for treatment and/or prevention of neurological disorders, for simultaneous, sequential, or separate use.

The term “interferon”, as used in the present patent application, is intended to include any molecule defined as such in the literature, comprising for example any kinds of IFNs mentioned in the above section “Background of the Invention”. The interferon may preferably be human, but also derived from other species, as long as the biological activity is similar to human interferons, and the molecule is not immunogenic in man.

In particular, any kinds of IFN-α, IFN-β and IFN-γ are included in the above definition. IFN-β is the preferred IFN according to the present invention.

The term “interferon-beta (IFN-β)”, as used in the present invention, is intended to include human fibroblast interferon, as obtained by isolation from biological fluids or as obtained by DNA recombinant techniques from prokaryotic or eukaryotic host cells as well as its salts, functional derivatives, variants, analogs and fragments.

Of particular importance is a protein that has been derivatized or combined with a complexing agent to be long lasting. For example, PEGylated versions, as mentioned above, or proteins genetically engineered to exhibit long lasting activity in the body, can be used according to the present invention.

The term “derivatives” is intended to include only those derivatives that do not change one amino acid to another of the twenty commonly occurring natural amino acids.

Interferons may also be conjugated to polymers in order to improve the stability of the proteins. A conjugate between Interferon β and the polyol Polyethlyenglycol (PEG) has been described in WO99/55377, for instance.

In another preferred embodiment of the invention, the interferon is Interferon-β (IFN-β), and more preferably IFN-β1a.

SDF-1 is preferably used simultaneously, sequentially, or separately with the interferon.

In a preferred embodiment of the present invention, SDF-1 is used in an amount of about 0.001 to 1 mg/kg of body weight, or about 0.01 to 10 mg/kg of body weight or about 9, 8, 7, 6, 5, 4, 3, 2 or 1 mg/kg of body weight or about 0.1 to 1 mg/kg of body weight.

The invention further relates to the use of a nucleic acid molecule for manufacture of a medicament for the treatment and/or prevention of a neurological disease, wherein the nucleic acid molecule comprises a nucleic acid sequence of SEQ ID NO: 6 or a nucleic acid sequence encoding a polypeptide comprising an amino acid sequence selected from the group consisting of:
(a) polypeptide comprising amino acids of SEQ ID NO: 1
(b) a polypeptide comprising amino acids of SEQ ID NO: 4
(c) a polypeptide comprising amino acids of SEQ ID NO: 7
(d) a polypeptide of (a) to (c) further comprising a signal sequence, preferably amino acids of SEQ ID NO: 5
(e) a mutein of any of (a) to (d), wherein the amino acid sequence has at least 40% or 50% or 60% or 70% or 80% or 90% identity to at least one of the sequences in (a) to (c);
(f) a mutein of any of (a) to (d) which is encoded by a DNA sequence which hybridizes to the complement of the native DNA sequence encoding any of (a) to (c) under highly stringent conditions;
(g) a mutein of any of (a) to (d) wherein any changes in the amino acid sequence are conservative amino acid substitutions to the amino acid sequences in (a) to (c);
(h) a salt or an isoform, fused protein, functional derivative, or active fraction of any of (a) to (d).

The nucleic acid may e.g. be administered as a naked nucleic acid molecule, e.g. by intramuscular injection.

It may further comprise vector sequences, such as viral sequence, useful for expression of the gene encoded by the nucleic acid molecule in the human body, preferably in the appropriate cells or tissues.

Therefore, in a preferred embodiment, the nucleic acid molecule further comprises an expression vector sequence. Expression vector sequences are well known in the art, they comprise further elements serving for expression of the gene of interest. They may comprise regulatory sequence, such as promoter and enhancer sequences, selection marker sequences, origins of multiplication, and the like. A gene therapeutic approach is thus used for treating and/or preventing the disease. Advantageously, the expression of SDF-1 will then be in situ.

In a preferred embodiment, the expression vector is a lentiviral derived vector. Lentiviral vectors have been shown to be very efficient in the transfer of genes, in particular within the CNS. Other well established viral vectors, such as adenoviral derived vectors, may also be used according to the invention.

A targeted vector may be used in order to enhance the passage of SDF-1 across the blood-brain barrier. Such vectors may target for example the transferrin receptor or other endothelial transport mechanisms.

In a preferred embodiment of the invention, the expression vector may be administered by intramuscular injection.

The use of a vector for inducing and/or enhancing the endogenous production of SDF-1 in a cell normally silent for expression of SDF-1, or which expresses amounts of SDF-1 which are not sufficient, are also contemplated according to the invention. The vector may comprise regulatory sequences functional in the cells desired to express SDF-1. Such regulatory sequences may be promoters or enhancers, for example. The regulatory sequence may then be introduced into the appropriate locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

The invention further relates to the use of a cell that has been genetically modified to produce SDF-1 in the manufacture of a medicament for the treatment and/or prevention of neurological diseases.

The invention further relates to a cell that has been genetically modified to produce SDF-1 for manufacture of a medicament for the treatment and/or prevention of neurological diseases. Thus, a cell therapeutic approach may be used in order to deliver the drug to the appropriate parts of the human body.

The invention further relates to pharmaceutical compositions, particularly useful for prevention and/or treatment of neurological diseases, which comprise a therapeutically effective amount of SDF-1 and a therapeutically effective amount of an interferon and/or osteopontin and/or clusterin optionally further a therapeutically effective amount of an immunosuppressant.

The definition of “pharmaceutically acceptable” is meant to encompass any carrier, which does not interfere with effectiveness of the biological activity of the active ingredient and that is not toxic to the host to which it is administered, or that can increase the activity. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, intrathecal, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo.

In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol (PEG), as described in the PCT Patent Application WO 92/13095.

The therapeutically effective amounts of the active protein(s) will be a function of many variables, including the type of protein, the affinity of the protein, any residual cytotoxic activity exhibited by the antagonists, the route of administration, the clinical condition of the patient (including the desirability of maintaining a non-toxic level of endogenous SDF-1 activity).

A “therapeutically effective amount” is such that when administered, the SDF-1 exerts a beneficial effect on the neurological disease. The dosage administered, as single or multiple doses, to an individual will vary depending upon a variety of factors, including SDF-1 pharmacokinetic properties, the route of administration, patient conditions and characteristics (sex, age, body weight, health, size), extent of symptoms, concurrent treatments, frequency of treatment and the effect desired.

As mentioned above, SDF-1 can preferably be used in an amount of about 0.001 to 1 mg/kg of body weight, or about 0.01 to 10 mg/kg of body weight or about 9, 8, 7, 6, 5, 4, 3, 2 or 1 mg/kg of body weight or about 0.1 to 1 mg/kg of body weight.

The route of administration, which is preferred according to the invention, is administration by subcutaneous route. Intramuscular administration is further preferred according to the invention.

In further preferred embodiments, SDF-1 is administered daily or every other day.

The daily doses are usually given in divided doses or in sustained release form effective to obtain the desired results. Second or subsequent administrations can be performed at a dosage which is the same, less than or greater than the initial or previous dose administered to the individual.

According to the invention, SDF-1 can be administered prophylactically or therapeutically to an individual prior to, simultaneously or sequentially with other therapeutic regimens or agents (e.g. multiple drug regimens), in a therapeutically effective amount, in particular with an interferon. Active agents that are administered simultaneously with other therapeutic agents can be administered in the same or different compositions.

The invention further relates to a method for treating a neurological disease comprising administering to a patient in need thereof an effective amount of SDF-1, or of an agonist of SDF-1 activity, optionally together with a pharmaceutically acceptable carrier.

A method for treating a neurological disease comprising administering to a patient in need thereof an effective amount of SDF-1, or of an agonist of SDF-1 activity, and an interferon, optionally together with a pharmaceutically acceptable carrier, is also within the present invention.

A method for treating a neurological disease comprising administering to a patient in need thereof an effective amount of SDF-1, or of an agonist of SDF-1 activity, and osteopontin, optionally together with a pharmaceutically acceptable carrier, is also within the present invention.

A method for treating a neurological disease comprising administering to a patient in need thereof an effective amount of SDF-1, or of an agonist of SDF-1 activity, and clusterin, optionally together with a pharmaceutically acceptable carrier, is also within the present invention.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning of a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

Human recombinant chemokines SDF-1α and SDF-1α variant were produced in house. The coding sequences (SEQ ID NO: 1 for SDF-1α and SEQ ID NO: 4 for SDF-1α variant) were cloned into Nde1/BamHI site of pET20b+vector and expressed in E. Coli cells.

### Example 1

### SDF-1 and SDF-1 Variant Activity Mixed Cortical Cultures Treated with LPS

### Introduction

Although considered an immunologically privileged site, the CNS can display significant inflammatory responses, which may play a role in a number of neurological diseases. Microglia appear to be particularly important for the initiating and sustaining of CNS inflammation. These cells exist in a quiescent form in the normal CNS, but acquire macrophage-like properties (including active phagocytosis, upregulation of proteins necessary for antigen presentation and production of proinflammatory cytokines) after stimulation by infections or T cells.

This inflammatory environment in vitro and in vivo can be mimicked by lipopolysaccharide (LPS), a component of the outer membranes of gram negative bacteria. LPS is the best characterised example of innate recognition that leads to a robust inflammatory response by phagocytic cells via the Toll receptor4. LPS has been widely used in the field to activate microglia in pure, co- or mixed cultures. Low levels of LPS induce cytokine release without inducing cell death, higher doses can induce oligodendrocyte or neuronal degeneration in vitro (Lehnardt et al., 2002; Sadir et al., 2001) and in vivo (Lehnardt et al., 2003; Sadir et al., 2001).

### Materials and Methods

Primary Mixed Cortical Cultures Preparation

Culturing of primary cells was performed as described (Lubetzki et al., 1993) using brain tissue from embryos isolated from NMRI mice at 16 days post-coitum. Cerebral hemispheres were dissected from embryo brains, dissociated via trypsin digestion and the single cell suspension was seeded at 5×10⁴ cells in 50 μl myelination medium per well onto BioCoat® poly-L-lysine coated 96-well plates (356516, Becton Dickinson). The myelination medium consisted of Bottenstein-Sato medium (Bottenstein and Sato, 1979; Sadir et al., 2001), supplemented with 1% FCS, 1% penicillin-streptomycin solution (Seromed) and recombinant platelet-derived growth factor AA (PDGF-AA, R&D Systems) at 10 ng/mL.

Treatment of Primary Mixed Cortical Cultures with LPS: Assay Set-Up

For the set up of cytokines release from primary mixed cortical cultures stimulated with LPS, cultures were grown at 37° C. and 10% CO₂ for 14 days and were then stimulated for 48 hours with increasing concentrations of LPS (0, 0.5, 1, 2.5, 5 ng/ml).

After 48 hours of LPS stimulation, 80 μl of supernatants were collected and frozen at −80° C. prior to content analysis of:
cytokine release (TNF-α and IL-6), analysed via CBA mouse inflammation kit (BD Biosciences 552364)SDF-1
SDF-1α using the sandwich ELISA set up in house and described here below.
cell viability assessed using an MTS assay (Promega G5421; Non-Radioactive Cell Proliferation Assay that measures mitochondrial activity through the formation of an insoluble formazan salt that has been shown to correlate to cell density).

SDF-1α ELISA

A sandwich ELISA for quantification of SDF-1α levels in mixed cortical cultures was set up in house. For coating 100 μl/well of monoclonal anti-mouse SDF-1 (1:500 R&D Systems Inc, Minneapolis, USA) was used, 100 μl/well of biotinylated polyclonal anti-mouse IgG (1:400 R&D Systems Inc, Minneapolis, USA) was used as secondary antibody and 100 μl/well of extravidin-conjugated horseradish peroxidase (1:5000 Sigma, St. Louis, Mo., USA). Recombinant mouse SDF-1 (2000 to 10 ng/ml R&D Systems Inc, Minneapolis, USA) was used to perform the standard curve. For visualization, 100 μl/well of substrate reagent pack a mixture of stabilized hydrogen peroxide and tetramethylbenzidine (R&D Systems Inc, Minneapolis, USA) was used. Optical density was measured using a fluoroplate reader (Labsystems Multiskan EX) at 450 nm.

SDF-1α and SDF-1α Variant Effects on Cytokine Expression in LPS Stimulated Cultures

For testing the effects of SDF-1α and SDF-1α variant (as defined in SEQ ID NO: 4) on LPS stimulated cultures, cells were allowed to grow for two weeks. At day 14 cells were pre-incubated with increasing concentrations (0.001, 0.1 and 10 ng/ml) of the corresponding proteins into 25 μl of medium for three hours at 37° C. and 10% CO₂. LPS was then supplemented to the cells at the concentration of 5 ng/ml into 25 μl of medium to obtain a final volume of 100 μl and incubated for 48 hours. Supernatants were collected at day 16 and the levels of TNF-α and IL-6 (the major cytokines released by activated microglia) were measured via specific ELISAs purchased from R&D systems (DuoSet mouse TNF-α ELISA DY410, mouse IL-6 ELISA DY406).

Two control molecules, dexamethasone and mouse IL-10, which have been shown to inhibit cytokine release from activated microglia, were used.

Data Analysis

Global analysis of the data was performed using one-way ANOVA. Dunnett's test was used further, and data were compared to the “untreated cells”. The level of significance was set at a: p<0.001; b or **: p<0.01; c or *: p<0.05; d: p<0.1. The results were expressed as mean ±standard error of the mean (s.e.m.).

### Results

Assay Set Up

TNF-α, IL-6 secretion was induced by LPS at 2.5 and 5 ng/ml and both doses where not toxic in the complex cultures. In addition the various concentrations of LPS (0, 0.5, 1, 2.5, 5 ng/ml) did not influence endogenous SDF-1α levels (results not shown).

SDF-1α and SDF-1αVariant

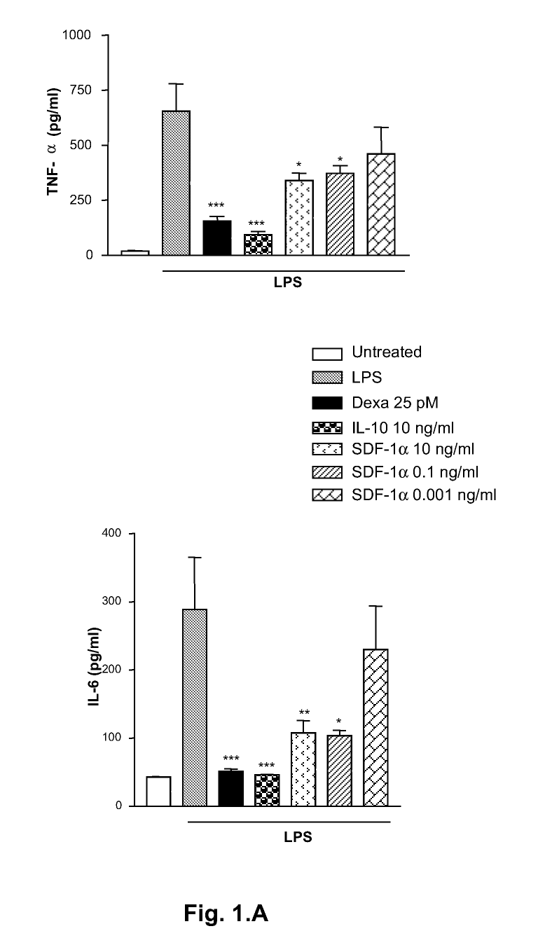

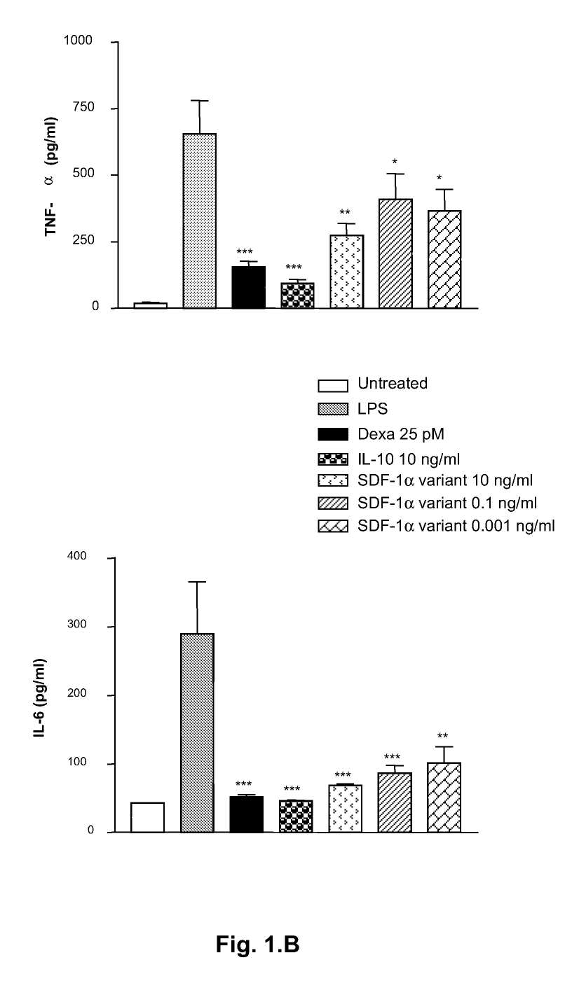

The results showed, that IL-10 at 10 ng/ml and Dexamethasone (25 pM) down regulated TNF-α and IL-6 as compared to untreated cells. Both SDF-1α and SDF-1α variant significantly decreased the levels of TNF-α and IL-6 secretion in the mixed cortical cultures after stimulation with LPS as compared to untreated cells and with a best concentration of 10 ng/ml (FIG. 1A and 1B).

### Conclusions

The mixed cortical cultures constitute a complex system that includes several neuro-epithelial cell types including astrocytes, microglia, neurons and oligodendrocytes. The non GAG binding mutant of SDF-1α, SDF-1-α variant, decreased TNF-α and IL-6 in a similar manner as SDF-1α indicating that GAG mutation does not affect SDF-1α binding to its receptor CXCR4.

The inhibition of cytokines seen with SDF-1α and SDF-1α variant in LPS treated mixed cortical cultures might be due to a direct action of SDF-1 on microglia or an indirect effect on CXCR4 receptor expressing astrocytes or neurons.

According to its clinical course, MS can be classified into several categories, stratifying MS patients with different patterns of disease activity. Patients with only rare relapses followed by full recovery of their disease are considered to have benign MS. Relapsing-Remitting MS (RRMS), the most common form of MS, is observed in 85-90% of MS patients and is characterized by recurrent relapses followed by recovery phases with residual deficits. The attacks are likely to be caused by the traffic of myelin-reactive T cells into the CNS, causing acute inflammation. Over time, the extent of recovery from relapses is decreased and baseline neurological disability increases. Ultimately, approximately 40% of RRMS patients no longer have attacks but develop a progressive neuro-degenerative secondary disorder related to chronic CNS inflammation, known as Secondary Progressive MS (SPMS) (Confavreux et al., 2000). The evolution to this secondary progressive form of the disease is associated with significantly fewer active lesions and a decrease in brain parenchymal volume. While earlier RRMS is sensitive to immunosuppression, the responsiveness to immunotherapy decreases in SPMS and may even disappear in late forms. Therefore, it could be hypothesized that RRMS and SPMS are a continuum rather than two diseases, where acute inflammatory events early on lead to the secondary induction of a neurodegenerative process.
The Primary Progressive form of MS (PPMS) is characterized from the onset by the absence of acute attacks and instead involves a gradual clinical decline. Clinically, this form of the disease is associated with a lack of response to any form of immunotherapy. Little is known about the pathobiology of Primary Progressive Multiple Sclerosis however, postmortem studies suggest that neuro-degeneration is predominant over inflammation in these patients. Interestingly grey matter damage predicts the evolution of primary progressive MS by being the strongest paraclinical predictor of subsequent worsening of disabilty (Rovaris 2006). Microglia activation in grey matter might contribute to accelerated neuronal loss and brain atrophy development. Therefore SDF-1alpha and SDF-1 variants may have a potential in treating primary progressive MS, due to their potential to regulate microglia activation and neuronal survival. Some of the pathophysiological mechanisms leading to neuronal loss might be overlaping in primary and secondary MS forms.

### Example 2

### SDF-1α Variant Effect on Leukocytes Recruitment in an In Vivo Model of Peritoneal Cell Recruitment

The major role of chemokines is to control migration of specific leukocyte populations during inflammatory responses and immune surveillance. Chemokines exert their biological effects by binding to seven transmembrane G protein-coupled receptors. They can also bind both soluble glycosaminoglycans (GAGs) as well as GAGs on cell surfaces which enhance local concentrations of chemokines, promoting their oligomerization and facilitating their presentation to the receptors. It has recently been demonstrated that chemokine interaction with GAGs is required for their chemotactic function in vivo.

### Material and Methods

8-12 week old, female Balb/C mice (Janvier, France) were injected intra peritoneally (i.p.) with 200 μl NaCl (0.9%, LPS free) or chemokine 4 μg (WT SDF-1α or SDF-1α variant according to SEQ ID NO:4 diluted in 200 μl NaCl (0.9%, LPS free). At 4 post injection of WT or mutant SDF-1α, mice were sacrificed by CO₂ asphyxiation, the peritoneal cavity was washed with 3×5 ml ice cold PBS and the total lavage was pooled for individual mice. Total cells collected were counted by haemocytometer (Neubauer, Germany).

### Results

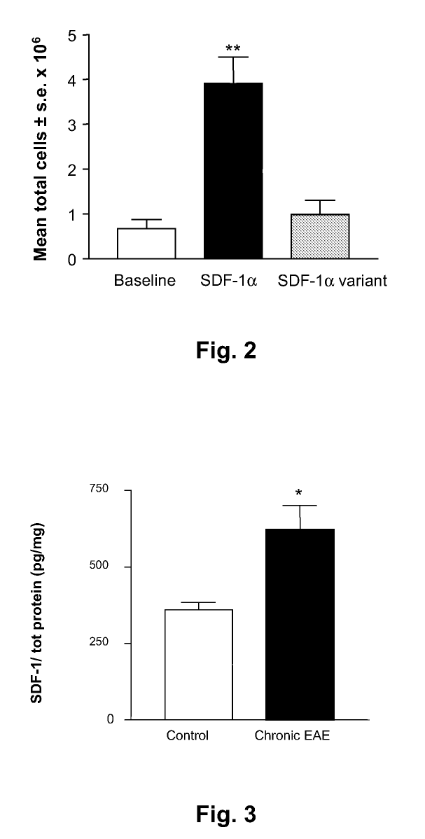

SDF-1α injected intra peritoneally recruits leukocytes. SDF-1α variant did not recruit leukocytes, showing that the in vivo GAG binding activity is lost by the mutation in the SDF-1α variant (see FIG. 2).

### Conclusions

The SDF-1α variant (GAG binding defective mutant of SDF-1) does not show leukocyte recruitment activity in vivo.

### Example 3

### SDF-1α Quantification in EAE Spinal Cord (Chronic)

### Introduction

SDF-1α expression was quantified in spinal cords dissected from mice afflicted with EAE induced by MOG peptide at chronic phase. The experimental autoimmune encephalomyelitis (EAE) model is a murine chronic demyelinating model and is an established animal model of multiple sclerosis (MS). The used method for the induction of EAE in mouse is adapted from the protocol published by Sahrbacher et al. (Sahrbacher et al., 1998).

### Material and Methods

Spinal Cord Sampling

Spinal cords were dissected from mice afflicted with EAE 4 weeks after the disease onset i.e. presence of tail paralysis as clinical sign. Mice were perfused with cold PBS and spinal cords were dissected out into triple detergent buffer (50 mM Tris, pH 8.0, 150 mM NaCl, 0.02% NaN₃, 0.1% SDS, 1% Nonidet P-40, 0.5% sodium deoxycholate) containing a protease inhibitor cocktail (Roche Molecular Biochemicals, 1836170, 1 tablet per 10 ml buffer). 100 μl of buffer was used per mg tissue obtained. Tissue samples were stored in plastic eppendorf tubes at −20° C. prior to preparation via homogenization and subsequent analysis.

Analysis of SDF-1α Content of Spinal Cord

Spinal cord were defrosted and homogenized in triple detergent buffer using a polytron. Protein levels in samples were quantified via BCA Protein Content Assay (Pierce Biotechnology, Rockford Ill. 61105, USA) prior to SDF-1 α content analysis using the ELISA described in the material and methods section of Example 1 above.

### Results

FIG. 3 shows an upregulation of SDF-1α in spinal cord tissue of EAE animals in the chronic phase of EAE.

### Conclusions

The up-regulation of SDF-1α protein in EAE spinal cord extracts from chronic MOG EAE phases, suggests a role for SDF-1α in neuro-inflammation other than inflammatory cell recruitment.

### Example 4

### Protective Effect of SDF-1α on Neuropathy Induced by Sciatic Nerve Crush

### Introduction

The present study was carried out to evaluate nerve regeneration and remyelination in mice treated with SDF-1α at different doses. A positive effect of SDF-1α on neuronal and axonal (sensory and motor neurons) survival and regeneration, or on myelination or macrophage inflammation, may lead to restoration of motor function. The regeneration can be measured according to the restoration of sensorimotor functions, which can be evaluated by electrophysiological recordings.

### Materials and Methods

Animals

Thirty 8 week-old females C57bl/6 RJ mice (Elevage Janvier, Le Genest-St-Isle, France) were used. They were divided into 6 groups (n=6):
(a) nerve crush/Vehicle (Saline/0.02% BSA);
(b) nerve crush/SDF-1α (3 μg/kg);
(c) nerve crush/SDF-1α (10 μg/kg);
(d) nerve crush/SDF-1α (30 μg/kg);
(e) nerve crush/SDF-1α (100 μg/kg);
(f) nerve crush/IL-6 (30 μg/kg).

The animals were group-housed (6 animals per cage) and maintained in a room with controlled temperature (21-22° C.) and a reversed light-dark cycle (12 h/12 h) with food and water available ad libitum. All experiments were carried out in accordance with institutional guidelines.

Lesion of the Sciatic Nerve

The animals were anaesthetized by inhalation of 3% Isoflurane (Baxter). The right sciatic nerve was surgically exposed at mid thigh level and crushed at 5 mm proximal to the trifurcation of the sciatic nerve. The nerve was crushed twice for 30s with a haemostatic forceps (width 1.5 mm; Koenig; Strasbourg; France) with a 90-degree rotation between each crush.

Planning of Experiments and Pharmacological Treatment

Electromyographical (EMG) testing was performed once before the surgery day and each week during 3 weeks following the operation.

The day of nerve crush surgery was considered as dpl 0 (dpl=day post lesion). No test was performed during the 4 days following the crush.

From the day of nerve injury to the end of the study, SDF-1α, IL-6 or Vehicle were administered daily by subcutaneous injections (s.c.) route, 5 days per week.

Electrophysiological Recording

Electrophysiological recordings were performed using a Neuromatic 2000M electromyograph (EMG) (Dantec, Les Ulis, France). Mice were anaesthetized by inhalation of 3% Isofluran® (Baxter). The normal body temperature was maintained using a heated operating table (Minerve, Esternay, France).

Compound muscle action potential (CMAP) was measured in the gastrocnemius muscle after a single 0.2 ms stimulation of the sciatic nerve at a supramaximal intensity (12.8 mA). The amplitude (mV) and the latency (ms) of the action potential were measured on the operated leg. The measures was also registered on the contralateral (uncrushed) leg of Vehicle treated animals (Baseline). The amplitude is indicative of the number of active motor units, while the distal latency indirectly reflects motor nerve conduction and neuromuscular transmission velocities, which depends in part on the degree of myelination.

Data Analysis

Global analysis of the data was performed using one-way ANOVA. Dunnett's test was used further, and data were compared to the “vehicle” control. The level of significance was set at a: p<0.001; b or **: p<0.01; c or *: p<0.05; d: p<0.1. The results were expressed as mean ±standard error of the mean (s.e.m.).

Electrophysiological Measurements

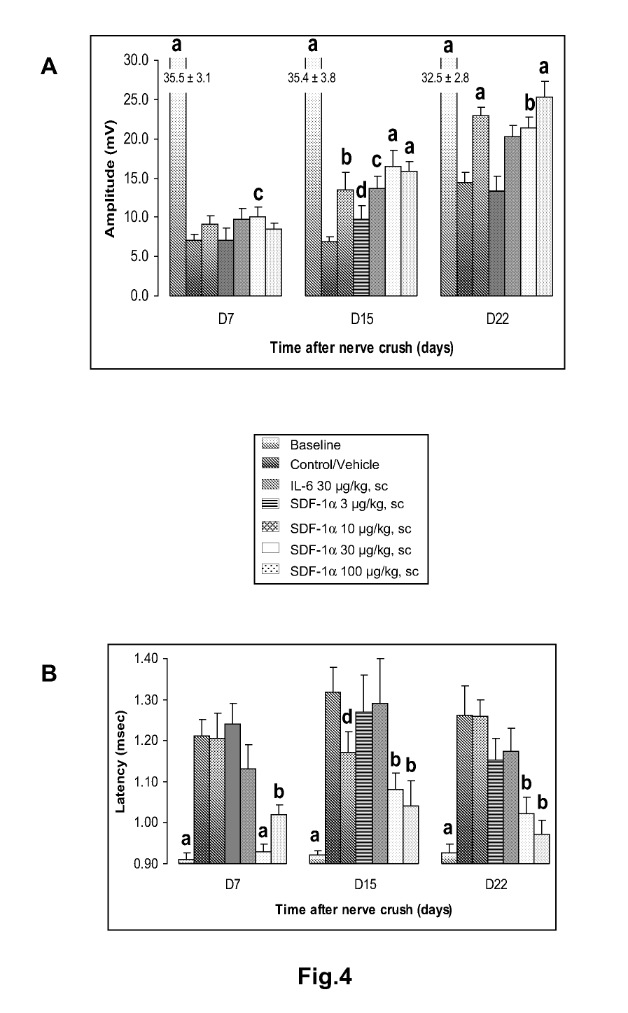

Amplitude of the Compound Muscular Action Potential (FIG. 4.A):

No significant change in the CMAP amplitude throughout the study was observed on the contralateral (uncrushed) legs of vehicle treated animals (Baseline). In contrast, crush of the sciatic nerve induced a dramatic decrease in the amplitude of CMAP with a decrease in the Vehicle treated group of about 80% at dpl7 and dpl15, when compared to the respective Baseline levels. When mice were treated with SDF-1α, at 30 μg/kg or μ/kg, or IL-6 at 30 μ/kg, they demonstrated an increase (about 1.5 times) in the CMAP amplitude, as compared to the levels in untreated mice, and this effect was significant at 15 dpl and 22dpl.

Latency of the Compound Muscular Action Potential (FIG. 4.B):

There was no deterioration of CMAP latency on the contralateral (uncrushed) legs of vehicle treated animals throughout the study. In contrast, muscles on the crushed side showed greater CMAP latency than the Baseline. In mice treated with SDF-1α, the CMAP latency value was significantly reduced as compared to the one of Vehicle treated mice. At day 7, this effect could be observed after treatment with 30 μg/kg and 100 μg/kg of SDF-1α but not with 30 μg/kg

of IL-6. At dpl 15 and 22, a significant effect was still obtained with 30 μg/kg and 100 μg/kg (but not with 3 or 10 μg/kg) of SDF-1α. SDF-1α (30 μg/kg) is more potent than IL-6 (30 μg/kg).

### Conclusions

The nerve-crush model is a very dramatic model of traumatic nerve injury and peripheral neuropathy. Immediately after the nerve crush most of the fibers having a big diameter are lost, due to the mechanical injury, leading to the strong decrease in the CMAP amplitude. The CMAP latency is not immediately affected but shows an increase at 15 days due to additional degeneration of small diameter fibers by secondary, immune mediated degeneration (macrophages, granulocytes). The CMAP duration is increased at dpl 7 and peaks at dpl 15.

SDF-1α restores function after peripheral nerve crush (CMAP latency). It also showed a protective effect in the nerve crush model in mice on all parameters measured. In summary, SDF-1α was as effective as the reference molecule used in this study, IL-6.

### Example 5

### Protective Effect of SDF-1α Variant on Neuropathy Induced by Sciatic Nerve Crush

The sciatic nerve crush model described in Example 4 above was carried out to test SDF-1α variant as defined in SEQ ID NO: 4 and the mice were divided into the following 2 groups (n=6):

(a) nerve crush operated/Vehicle (Saline/0.02% BSA);

(b) nerve crush/SDF-1α variant at 30 μg/kg s.c.

The measures registered on the contralateral leg of Vehicle treated animals were considered as Baseline values.

The SDF-1α variant used in this example and encoded by SEQ ID NO: 4 was expressed with an additional N terminal Methionine. The CMAP duration (time needed for a depolarization and a repolarization session) was also recorded.

### Results

Electrophysiological Measurements

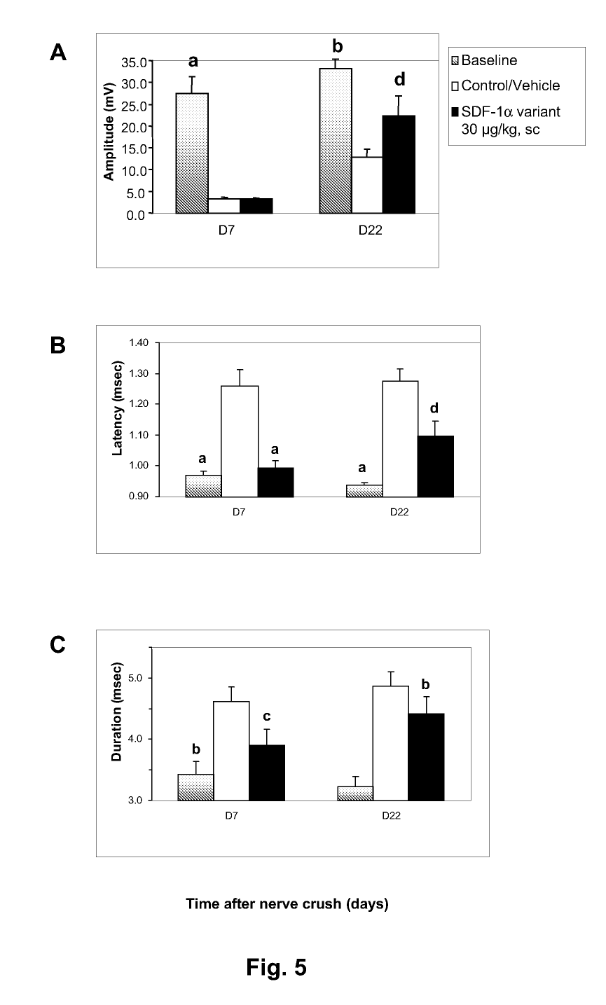

Amplitude of the Compound Muscular Action Potential (FIG. 5.A):

A significant increase in the CMAP amplitude was demonstrated at 22 dpl when mice were treated with SDF-1α variant.

Latency of the Compound Muscular Action Potential (FIG. 5.B):

In mice treated with SDF-1α variant, the CMAP latency value was significantly reduced as compared to the one of vehicle treated mice, especially at 7 dpl. A positive effect was still obtained at 22 dpl.

Duration of the Compound Muscular Action Potential (FIG. 5.C):

In mice treated with SDF-1α variant, the CMAP duration value was reduced as compared to the one of vehicle treated mice at 7 dpl and 22 dpl

### Conclusions

SDF-1α variant was shown to restore function after peripheral nerve crush (CMAP latency). It also showed a protective effect in the nerve crush model in mice on all parameters measured.

### Example 6

### Protective Effect of Met-SDF-1α on Neuropathy Induced by Sciatic Nerve Crush

The sciatic nerve crush model described in Example 4 above was carried out to test Met-SDF-1α (as defined in SEQ ID NO: 7) and the mice were divided into the following 2 groups (n=6):

(a) nerve crush operated/Vehicle (Saline/0.02% BSA);

(b) nerve crush/Met-SDF-1α variant at 100, 30, and 10 μg/kg s.c.

The measures registered on the contralateral leg of Vehicle treated animals were considered as Baseline values.

The CMAP duration (time needed for a depolarization and a repolarization session) was also recorded.

### Results

Electrophysiological Measurements

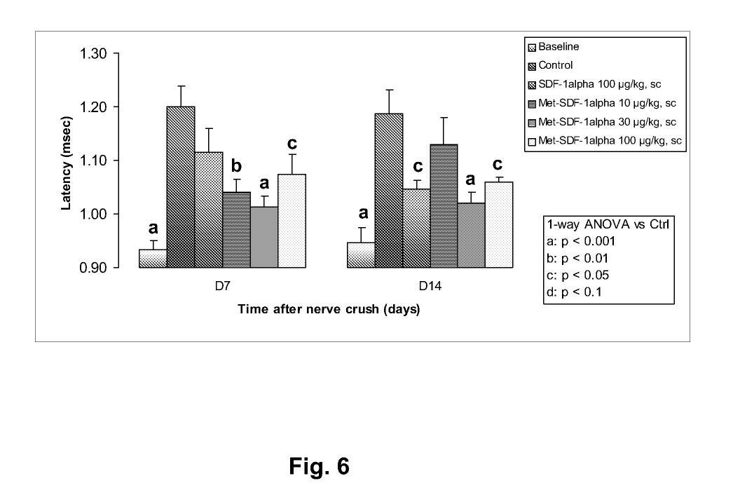

Latency of the Compound Muscular Action Potential (FIG. 6):

In mice treated with Met-SDF-1α, the CMAP latency value was significantly reduced at day 7 and day 14 after crush as compared to the one of vehicle treated mice.

### Conclusions

Met-SDF-1α was shown to restore function after peripheral nerve crush (CMAP latency) as well as SDF-1α.

### Example 7

### Protective Effect of SDF-1α in Diabetic Neuropathy

### Introduction

Diabetic neuropathy is the most common chronic complication of diabetes. The underlying mechanisms are multiple and appear to involve several interrelated metabolic abnormalities consequent to hyperglycemia and to insulin and C-peptide deficiencies. The most common early abnormality indicative of diabetic neuropathy is asymptomatic nerve dysfunction as reflected by decreased nerve conduction velocity (Dyck and Dyck, 1999). These changes are usually followed by a loss of vibration sensation in the feet and loss of ankle reflexes. Electrophysiological measurements often reflect fairly accurately the underlying pathology and changes in nerve conduction velocity correlate with myelination of nerve fibers (for review see Sima, 1994).

The streptozotocin (STZ) diabetic rat is the most extensively studied animal model of diabetic neuropathy. It develops an acute decrease in nerve blood flow (40%) and slowing of nerve conduction velocity (20%) (Cameron et al., 1991), followed by axonal atrophy of nerve fibers (Jakobsen, 1976). Demyelinating and degenerating myelinated fibers as well as axo-glial dysjunction are seen with long-lasting diabetes (Sima et al., 1988).

The primary goal of the present investigation was to explore the potential neuro- and gliaprotective effect of SDF-1α on the development of diabetic neuropathy in STZ-rats.

### Materials and Methods

Animals

Eight week-old male Sprague Dawley rats (Janvier, Le Genest Saint Isle, France) were randomly distributed in 6 experimental groups (n=10) as shown below.

TABLE IV

| | | | Treatment |
|---|---|---|---|
| | | Adminis- | period |
| | | tration | (days |
| Group (n = 10) | Treatments | routes | post-STZ) |
| Control/Vehicle | daily Vehicle | s.c. | 11 to 40 |
| STZ/Vehicle | daily Vehicle | s.c. | 11 to 40 |
| STZ/SDF-1α (10 μg/kg) | daily SDF-1α | s.c. | 11 to 40 |
| STZ/SDF-1α (30 μg/kg) | daily SDF-1α | s.c. | 11 to 40 |
| STZ/SDF-1α (100 μg/kg) | daily SDF-1α | s.c. | 11 to 40 |
| STZ/IL-6 (10 μg/kg) | daily IL-6 | s.c. | 11 to 40 |

They were group-housed (3 animals per cage) and maintained in a room with controlled temperature (21-22° C.) and a reversed light-dark cycle (12 h/12 h) with food and water available ad libitum. All experiments were carried out in accordance with institutional guidelines.

Induction of Diabetes and Pharmacological Treatment

Diabetes was induced by intravenous injection of a buffered solution of streptozotocin (Sigma, L'Isle d'Abeau Chesnes, France) at a dose of 55 mg/kg. STZ was prepared in 0.1 mol/l citrate buffer pH 4.5. Control group received an equivalent volume of citrate buffer. The day of STZ injection was considered as D0.

At D10 post-STZ, glycemia was monitored for each individual animal. Animals showing a value below 260 mg/dl were excluded from the study.

Treatment with SDF-1α, with IL-6 or their matched vehicle was performed on daily basis from D11 to D40.

SDF-1 α and IL-6 were prepared in saline solution (0.9% NaCl) containing 0.02% BSA.

Planning of Experiments
Day −7: baseline (EMG)
Day 0: induction by the streptozotocin
Day 7: glycemia monitoring
Day 11: Onset of the treatment
Day 20: Von Frey test
Day 25: EMG monitoring
Day 40: EMG monitoring and HP 52° C. test
Day 41: sciatic nerves and skin biopsy samples were taken off for the histomorphometric analysis.

Electromyography

Electrophysiological recordings were performed using electromyograph (Keypoint, Medtronic, Boulogne-Billancourt, France). Rats were anaesthetized by intraperitoneal injection (IP) of 60 mg/kg ketamine chlorhydrate (Imalgene 500®, Rhône Mérieux, Lyon. France) and 4 mg/kg xylazin (Rompum 2%, Bayer Pharma, Kiel, Germany). The normal body temperature was maintained at 30° C. with a heating lamp and controlled by a contact thermometer (Quick, Bioblock Scientific, Illkirch, France) placed on the tail surface.

Compound muscle action potential (CMAP) was recorded in the gastrocnemius muscle after stimulation of the sciatic nerve. A reference electrode and an active needle were placed in the hindpaw. A ground needle was inserted on the lower back of the rat. Sciatic nerve was stimulated with a single 0.2 ms pulse at a supramaximal intensity. The velocity of the motor wave was recorded.

Sensitive nerve conduction velocity (SNCV) was also recorded. The tail skin electrodes were placed as follows: a reference needle inserted at the base of the tail and an anode needle placed 30 mm away from the reference needle towards the extremity of the tail. A ground needle electrode was inserted between the anode and reference needles. The caudal nerve was stimulated with a series of 20 pulses (for 0.2 ms) at an intensity of 12.8 mA. The velocity was expressed in m/s.

Morphometric Analysis

Morphometric analysis was performed at the end of the study. The animals were anesthetized by IP injection of 60 mg/kg Imalgéne 500®. A 5 mm-segment of sciatic nerve was excised for histology. The tissue was fixed overnight with 4% glutaraldehyde (Sigma, L'Isle d'Abeau-Chesnes, France) solution in phosphate buffer solution (pH 7.4) and maintained in 30% sucrose at +4° C. until use. The nerve sample was fixed in 2% osmium tetroxide (Sigma) solution in phosphate buffer solution for 2 h, dehydrated in serial alcohol solution, and embedded in Epon. Embedded tissues were then placed at +70° C. during 3 days of polymerization. Transverse sections of 1.5 μm thickness were obtained using a microtome. They were stained with a 1% toluidine blue solution (Sigma) for 2 min, dehydrated and mounted in Eukitt.

Analysis was performed on the entire surface of the nerve section using a semi-automated digital image analysis software (Biocom, France). Once extraneous objects had been eliminated, the software reported the total number of myelinated fibers. The number of degenerated fibers was then counted manually by an operator. Myelinated fibers without axons, redundant myelin and fibers showing sheaths with too large thickness in respect to their axonal diameter were considered as fibers undergoing processes of degeneration. The number of non-degenerated fibers was obtained by subtraction of the number of degenerated fibers.

Morphological analysis was performed only on fibers considered as non-degenerated. For each fiber, the axonal and myelin sizes were reported in surface area (μm²). These two parameters were used to calculate the equivalent area of g-ratio (axonal diameter/fiber diameter) of each fiber (i.e., [A/(A+M)]^{0.5}, A=axonal area, M=myelin area), indicative of the relative myelin sheath thickness.

In addition, a 5-10 mm diameter area of skin was punch-biopsied from the hindpaw. Skin samples were immediately fixed overnight in paraformaldehyde at 4° C., incubated (overnight) in 30% sucrose in 0.1 M PBS for cryoprotection, embedded in OCT and frozen at −80° C. until cryocut.

50 μm-thick cryosections were then cut vertical to the skin surface with a cryostat. Free-floating sections were incubated for 7 days in a bath of rabbit anti-protein gene product 9.5 (1:10000; Ultraclone, Isle of Man, UK) at 4° C. The sections were then processed to reveal immunoreactivity according to the ABC peroxidase method. Briefly, they were incubated in for 1 h with biotinylated anti-goat antibody (1:200), then 30 min in the avidin biotinylated complex at room temperature. Peroxidase activity was visualized using DAB system. Sections were then counterstained with eosin or hematoxylin. Sections were dehydrated, clear with bioclear and mounted on eukitt. Photos of microscope fields were performed at 20× power magnification view using Nikon digital camera at focal distance of 12.9 mm. The number of intra-epidermal nerves on 3 microscope fields of 0.22 μm² (544×408 μm) each was counted by the experimenter on computer screen.

Data Analysis

Global analysis of the data was performed using one factor or repeated measure analysis of variance (ANOVA) and one-way ANOVA. When ANOVA indicated significant difference, Fisher Protected Least Significant Difference was used as post-hoc test to compare experimental groups with the group of diabetic rats treated with the vehicle. The level of significance was set at p≦0.05. Results are expressed as mean ±standard error of the mean (s.e.m.).

### Results

Body Weight

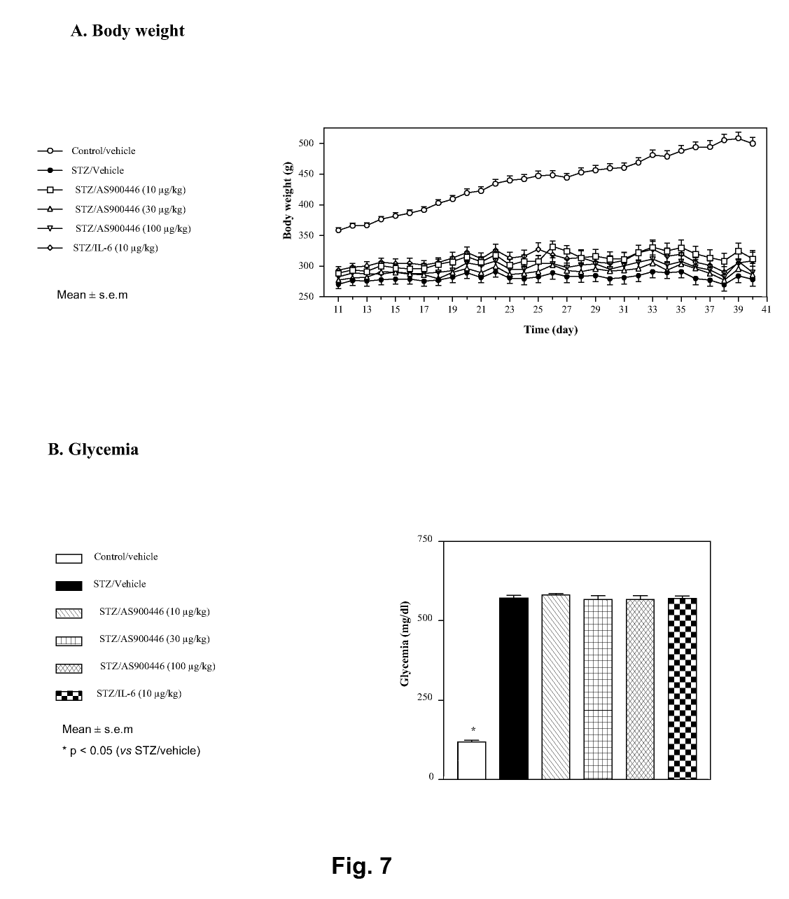

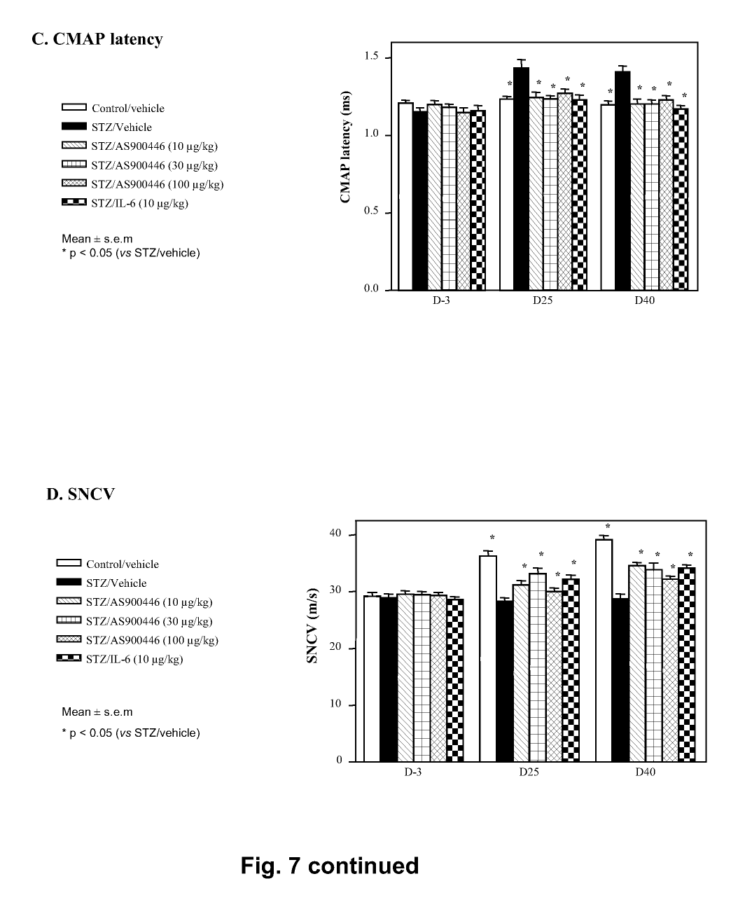

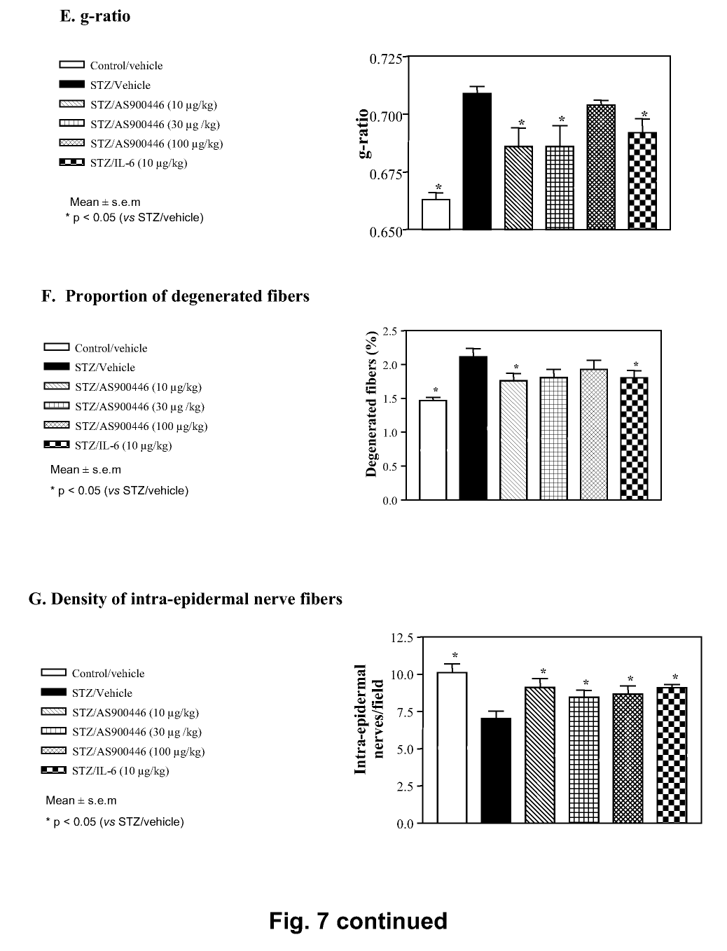

In contrast with non-diabetic rats showing a progressive growth, diabetic rats demonstrated a significant growth arrest (FIG. 7A).

Treatment with SDF-1α or with IL-6 was associated with slight but significant increase in the body weight of vehicle-treated diabetic rats.

Glycemia

At day 7 post-STZ, all rats that had received STZ showed glycemia 5 times higher than that of control rats (FIG. 7B).

Electrophysiological Measurements

1. Latency of the Compound Muscle Action Potential

The CMAP latency was significantly extended in diabetic rats on D25 as compared to that of non-diabetic rats (FIG. 7C). Treatment with SDF-1α or with IL-6 induced a significant reduction in the CMAP latency of diabetic rats as compared to that of vehicle-treated diabetic rats.

Similar profile of results was observed at D40 post-STZ.

2. Sensory Nerve Conduction Velocity

At D25, vehicle-treated diabetic rats demonstrated a significantly reduced SNCV as compared to non-diabetic rats (FIG. 7D). Treatment with SDF-1α or with IL-6 significantly improved the SNCV performance of diabetic rats. The best effect was observed with the treatment doses of 10 and 30 μg/kg and was comparable with the one associated with IL-6 treatment.

Similar profile of results was observed at D40 post-STZ.

Morphometric Analysis

1. G-Ratio (Relative Myelin Thickness)

The g-ratio of diabetic rats receiving vehicle was significantly increased as compared to that of non-diabetic rats (FIG. 7E), suggesting a thinning of myelin sheath in diabetic rats. Treatment of diabetic rats with SDF-1α significantly reduced g-ratio as compared to STZ/Vehicle group, especially for the doses of 10 or 30 μg/kg. At the dose of 100 μg/kg, the reduction in g-ratio value did not reach the significance level.

IL-6 treatment also induced a significant reduction in the g-ratio value.

2. Number of Degenerated Fibers

Diabetic rats receiving vehicle showed significantly greater proportion of degenerated fibers than non-diabetic rats (FIG. 7F). Conversely, the proportion of non-degenerated fibers in diabetic rats was significantly reduced as compared to that of non-diabetic rats (FIG. 7F). Treatment of diabetic rats with SDF-1α showed reduction of degenerated fibers population. The best effect was associated with the lowest dose implemented (10 μg/kg) and reached the significance level.

A significantly reduced population of degenerated fibers was also observed in diabetic rats treated with IL-6.

As shown in FIG. 7G, diabetic rats receiving vehicle showed significantly reduced density of intra-epidermal nerve fibers compared to non-diabetic rats. Treatment of diabetic rats with SDF-1α was associated with significantly greater density of dermal nerve fibers than treatment with the vehicle. The observed effect was comparable with that induced by IL-6 treatment.

### Conclusions

In the present study, we wished to evaluate the neuro- and glia-protective effect of SDF-1α on the development of diabetes-related neuropathy. Investigations were conducted on STZ-induced diabetic related neuropathy in the rat. Similarly to the clinical setting of diabetic neuropathy, an impaired sensory nerve conduction detected as early as day 7 post-STZ is the first sign to indicate ongoing neuropathy in this model, which is in agreement with evidences of demyelination and/or axonal degeneration observed at later time-points (Andriambeloson et al., 2006). A previous study demonstrated that treatment of STZ-rats with low dose of IL-6 hampered the progression of neuropathy in this model without interfering with the development of glycemia (Andriambeloson et al., 2006).

In the present study, we found that chronic administration of SDF-1α (10, 30 and 100 μg/kg) improves the sensorimotor performance of diabetic rats (SNCV and CMAP latency scores) within about 2 weeks of treatment. The best effect was obtained with the treatment dose of 10 or 30 μg/kg, showing comparable efficiency as 10 μg/kg IL-6. In addition, SDF-1α treatment at these doses was found to markedly prevent the loss of myelin associated with this model. Since the quality of myelin sheath is an important component for optimal nerve conduction, preservation of the size of myelin sheath may, de facto, in part explain the improvement in nerve function of diabetic rats receiving SDF-1α treatment. Furthermore, it was also observed that SDF-1α reduces the population of fibers undergoing axonal degeneration in the sciatic nerve.

Similarly to the clinical setting of diabetic neuropathy showing correlation between the presence and the severity of neuropathy and degeneration of intra-epidermal nerve fibers from skin biopsy (Herrmann et al., 1999; Smith et al., 2001), STZ-induced diabetic neuropathy in rats also demonstrate that clinical signs of neuropathy in this animal model was strongly correlated with the reduction in the density of intra-epidermal nerve fibers. In line with the above findings, the present study showed that vehicle-treated diabetic rats demonstrate significant reduction in intra-epidermal nerve fibers density. This phenomenon was markedly prevented by the treatment with SDF-1α or with IL-6 and thus further supporting the neuroprotective effect of with SDF-1α regards to diabetes-induced nerve damage.

Altogether, the above findings indicate neuroprotective effect of SDF-1α treatment in the rat model of diabetic neuropathy. SDF-1α is an interesting candidate in the development of treatment therapy for clinical diabetic neuropathy.

### Example 8

### Protective Effect of SDF-1α in Neuropathic Pain

### Introduction

The most common precipitating cause of neuropathic pain is diabetes particularly where blood glucose control is poor. Approximately 2-24% of diabetes patients experience neuropathic pain. Diabetic neuropathic pain can occure either spontaneously, as a result of exposure to normally mildly painful stimuli (ie. Hyperalgesia), or to stimuli that are not normally perceived as being painful (ie. Allodynia). A number of anomalies in pain perception have been demonstrated in the streptozotocin model (Hounsom and Tomlinson, 1997) at early stage of diabetes. For example formalin-evoked flinching is exaggerated in STZ-rats as compared to control animals. In addition, the development of tactile allodynia has been reported in this animal model of diabetes (Calcutt et al., 1995, 1996). At later stage when hyperglycemia persists (Bianchi et al., 2004), extension of hot plate threshold has been reported as behavioral abnormality in diabetic rats.

### Materials and Methods

Animals

Eight week-old male Sprague Dawley rats (Janvier, Le Genest Saint Isle, France) were randomly distributed in 6 experimental groups (n=10) as shown below.

TABLE V

| | | | Treatment |
|---|---|---|---|
| | | Adminis- | period |
| | | tration | (days |
| Group (n = 10) | Treatments | routes | post-STZ) |
| Control/Vehicle | daily Vehicle | s.c. | 11 to 40 |
| STZ/Vehicle | daily Vehicle | s.c. | 11 to 40 |
| STZ/SDF-1α (10 μg/kg) | daily SDF-1α | s.c. | 11 to 40 |
| STZ/SDF-1α (30 μg/kg) | daily SDF-1α | s.c. | 11 to 40 |
| STZ/SDF-1α (100 μg/kg) | daily SDF-1α | s.c. | 11 to 40 |
| STZ/IL-6 (10 μg/kg) | daily IL-6 | s.c. | 11 to 40 |

They were group-housed (3 animals per cage) and maintained in a room with controlled temperature (21-22° C.) and a reversed light-dark cycle (12 h/12 h) with food and water available ad libitum. All experiments were carried out in accordance with institutional guidelines.

Induction of Diabetes and Pharmacological Treatment

Diabetes was induced by intravenous injection of a buffered solution of streptozotocin (Sigma, L'Isle d'Abeau Chesnes, France) at a dose of 55 mg/kg. STZ was prepared in 0.1 mol/l citrate buffer pH 4.5. The control group received an equivalent volume of citrate buffer. The day of STZ injection was considered as D0.

At D10 post-STZ, glycemia was monitored for each individual animal. Animals showing a value below 260 mg/dl were excluded from the study.

Treatment with SDF-1α with IL-6 or their matched vehicle was performed on daily basis from D11 to D40.

SDF-1α and IL-6 were prepared in saline solution (0.9% NaCl) containing 0.02% BSA.

Planning of Experiments

Day 0: induction by the streptozotocin

Day 7: glycemia monitoring

Day 11: Onset of the treatment

Day 20: Von Frey test

Day 40: EMG monitoring and HP 52° C. test

Von Frey Filament Test

The rat was placed on a metallic grid floor. The nociceptive testing was done by inserting the Von Frey filament (Bioseb, France) through the grid floor and applying it to the plantar surface of the hind paw. A trial consisted of several applications of the different von Frey filaments (at a frequency of 1-1.5 s). The Von Frey filaments were applied from filament 10 g to 180 g. The pressure that produces a brisk withdrawal of hind paw was considered as threshold value. Cuttoff value was set to 180 g.

Hot Plate 52° C. Test

The animal was placed into a glass cylinder on a hot plate adjusted to 52° C. The latency of the first reaction was recorded (licking, brisk movement of the paws, little leaps or a jump to escape the heat) with a cutoff time of 30 s.

### Results

Von Frey Filament

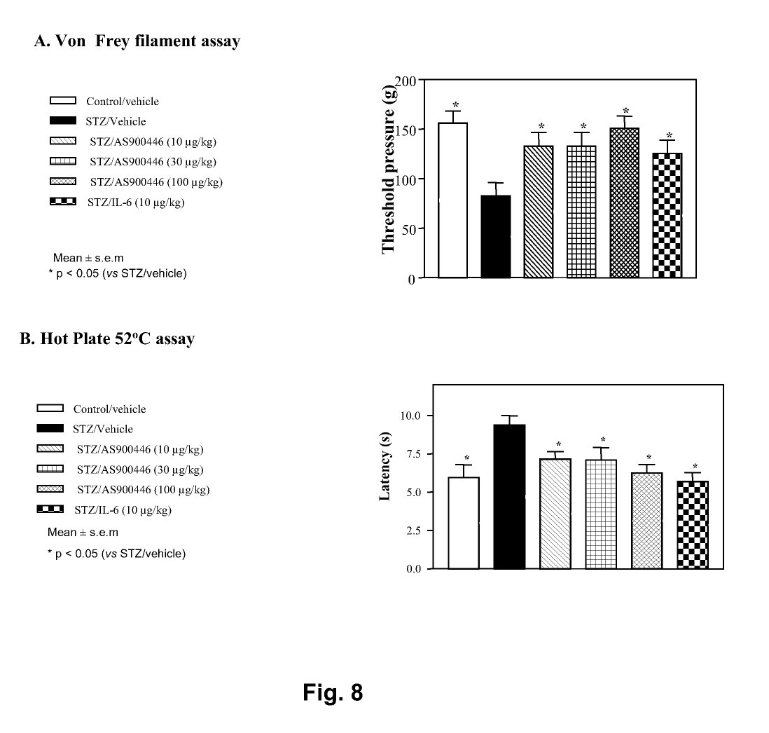

At day 20 post-STZ, vehicle-treated diabetic rats showed significantly lower threshold in Von Frey test than non-diabetic rats (FIG. 8A).

Treatment with SDF-1α or with IL-6 induced a significant increase in the threshold value of diabetic rats as compared to the score of vehicle-treated diabetic rats. The threshold values of SDF-1α or IL-6-treated rats were not statistically different to that of non-diabetic rats.

Hot Plate 52° C. Test

At D40 post-STZ, diabetic rats receiving vehicle treatment demonstrated significantly greater threshold latency in the hot plate test as compared to non-diabetic rats (FIG. 8B).

Treatment of diabetic rats with SDF-1α or with IL-6 significantly lowered the threshold latency of diabetic rats to a level statistically comparable with that of non-diabetic rats.

### Conclusions

Behaviors of rats in response to Von Frey filament (mechanical stimulation) and to heat (52° C.) were evaluated, at D20 and D40, respectively. In these two tests, diabetic rats treated with SDF-1α obviously showed behavior difference as compared to those treated with the vehicle and their score became comparable with that of non-diabetic rats.

These results seem to be in line with the electrophysiological and histological finding and suggest that SDF-1α can also be protective in neuropathic pain.

### Example 9

### Genetic Association Between SDF-1 Gene and Primary Progressive MS

### Materials and Methods

Collections of Patients and Controls

The study comprised one collection of unrelated patients with primary progressive MS (MSPP). All the subjects in the study were Caucasian from Italy. Patients and controls from Sardinia were discarded.

We included 197 patients with progressive course. 141 had a progression of neurological symptoms from the beginning of the disease, without relapses (Primary Progressive); 39 had a progressive course with superimposed relapses (Progressive Relapsing); 17 had a progressive course beginning many years after an isolated attack (Single-attack Progressive). The control population comprised 234 unrelated healthy controls from the same ethnic background as the case population.

The group of cases had a sex ratio of 1.05 (101 Females and 96 Males) and a mean age at onset of 39.2 [19-65] years. The group of controls included 234 individuals, with a sex ratio of 1.03 (119 Females and 115 Males) and a mean age of 40.4 [19-70] years.

Genotyping

Methods for Whole Genome Analysis: Affymetrix Method

250 ng (5 μl) of DNA from each sample was digested in parallel with 10 units of Nsp I and Sty I restriction enzymes (New England Biolabs, Beverly, Mass.) for 2 hours at 37° C. Enzyme specific adaptor oligonucleotides were then ligated onto the digested ends with T4 DNA Ligase for three hours at 16° C. After dilution with water, 5 μl of the diluted ligation reactions were subjected to PCR. PCR was performed with Titanium Taq DNA Polymerase (BD Biosciences, San Jose, Calif.) in the presence of 25 μM PCR primer 002 (Affymetrix), 350 μM each dNTP, 1 M Betaine (USB, Cleveland, Ohio), and 1× Titanium Taq PCR Buffer (BD Biosciences). Cycling parameters were as follows, initial

denaturation at 94° C. for 3 minutes, amplification at 94° C. for 30 seconds, 60° C. for 30 seconds and extension at 68° C. for 15 seconds repeated a total of 30 times, final extension at 68° C. for 7 minutes. PCR products from three reactions were combined and purified with the MinElute 96-well UF PCR purification plates (Qiagen, Valencia, Calif.) according to the manufacturer's directions. Samples were collected into microfuge tubes and spun at 16,000×g for 10 minutes.

The purified product was recovered from the tube taking special care not to disturb the white, gellike pellet of magnesium phosphate. PCR products were then verified to migrate at an average size between 200-800 bps using 2% TAE gel electrophoresis. Sixty micrograms of purified PCR products were then fragmented using 0.25 units of DNAse I at 37° C. for 35 minutes. Complete fragmentation of the products to an average size less than 180 bps was verified using 2% TAE gel electrophoresis. Following fragmentation, the DNA was end labeled with 105 units of terminal deoxynucleotidyl transferase at 37° C. for 2 hours. The labeled DNA was then hybridized onto the respective Mendel array at 49° C. for 18 hours at 60 rpm. The hybridized array was washed, stained, and scanned according to the manufacturer's (Affymetrix) instructions.

Genotype calls were obtained using the DM algorithm at a pValue of 0.33 followed by a batch analysis using the BRLMM algorithm following Affymetrix specifications.

SNP Filtering

SNPs have been filtered with the following criteria:

Missing genotypes rate must be <5%

The Minimum Allele Frequency (MAF) must be >1% in controls

The probability not to be at Hardy-Weinberg equilibrium must be <2% in controls

The SNP must be polymorphic in cases

Only SNPs from autosomal chromosomes were kept for analysis

Statistical Analysis

Method:

The FDR (false discovery rate) has been estimated with 10,000 permutations with the following univariate tests (using exact tests with Pearson's statistic) for each population:

Allelic test

Genotypic test

Minimum of allelic and genotypic test (abbreviated ‘min’)

Maximum of allelic and genotypic test (abbreviated ‘max’)

### Results

SNP Filtering and Genomic Coverage

Applying the filters defined above reduced the number of remaining SNPs as shown in Table VI:

TABLE VI

| Scan | #SNPs total | #SNPs after filtering | % remaining |
|---|---|---|---|
| MSPP cases vs. | 497,641 | 323,664 | 65% |
| MS controls | | | |

FDR

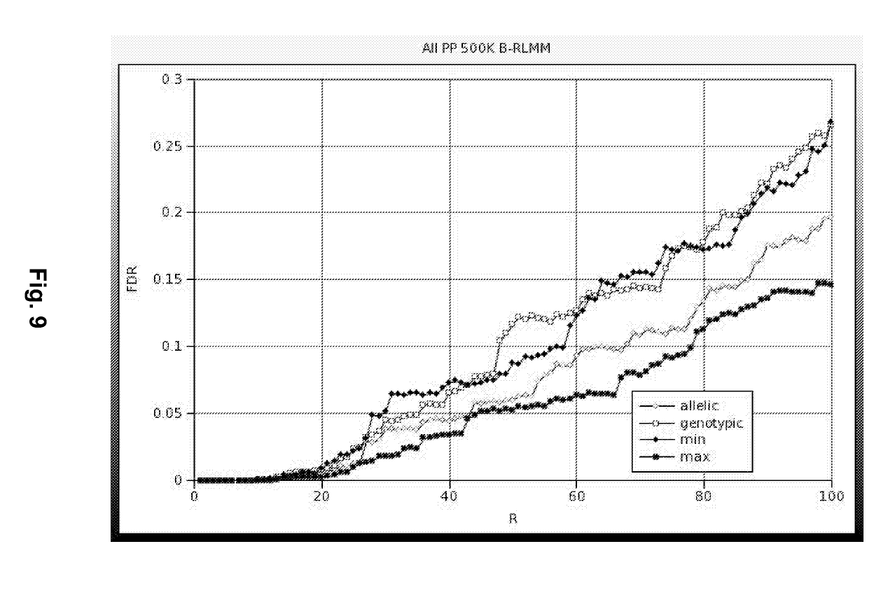

The FDR results are shown in FIG. 9

At a FDR threshold of 10%, the SNPs and genes were selected as shown in Table VII.

TABLE VII

| Scan | #SNPs | #BINs | #genes | #deserts |
|---|---|---|---|---|
| MSPP vs controls | 78 | 72 | 62 | 10 |

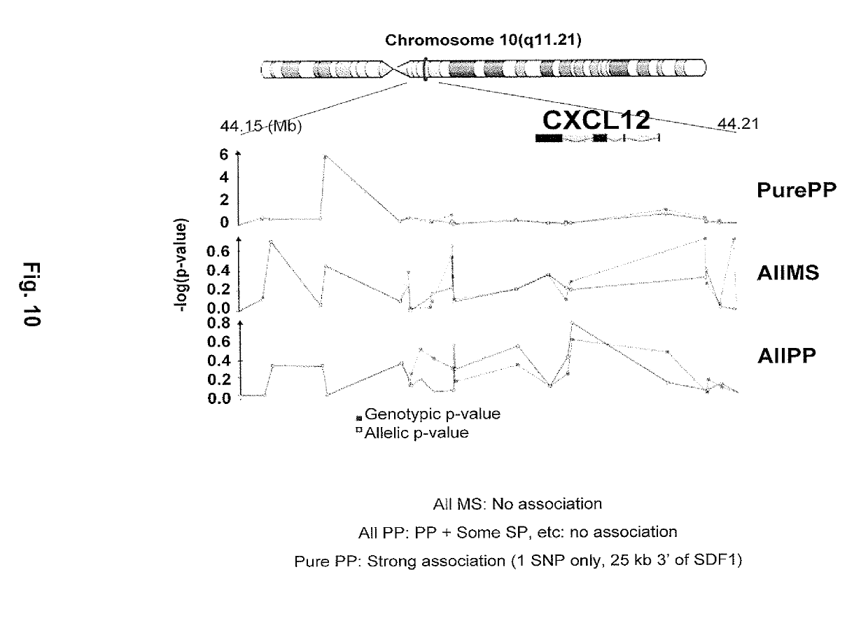

One SNP (SNP_A-2185631) was selected in the SDF-1 (CXCL2) gene (see FIG. 10.)

By looking at the contingency tables, we can see that the association comes from the differential distribution of allele C in cases and control populations.

TABLE VIII

MSPP vs controls

| Genotypes | cases | Controls |
|---|---|---|
| CC | 2.2% | 0.0% |
| CG | 22.8% | 7.4% |
| GG | 75.0% | 92.6% |

A detailed bioanalysis of the region around this SNP shows that it is located in an intron of recently discovered novel isoforms of SDF-1, as described in the following:

According to Ensembl, which only identified the two isoforms SDF-1 alpha and SDF-1 beta, SNP_A-2185631 is located 25 kb downstream of the SDF-1 (also known as CXCL12) gene. No gene is located nearer to SNP_A-2185631.

SDF-1 is located on chromosome 10 (44,192,517-44,200,551, NCBI build 35) and spans 8 kb.

An annotation of the genomic sequence has been performed in order to know if the SNP_A-2185631 could be related to SDF-1 gene or to another neighbouring gene.

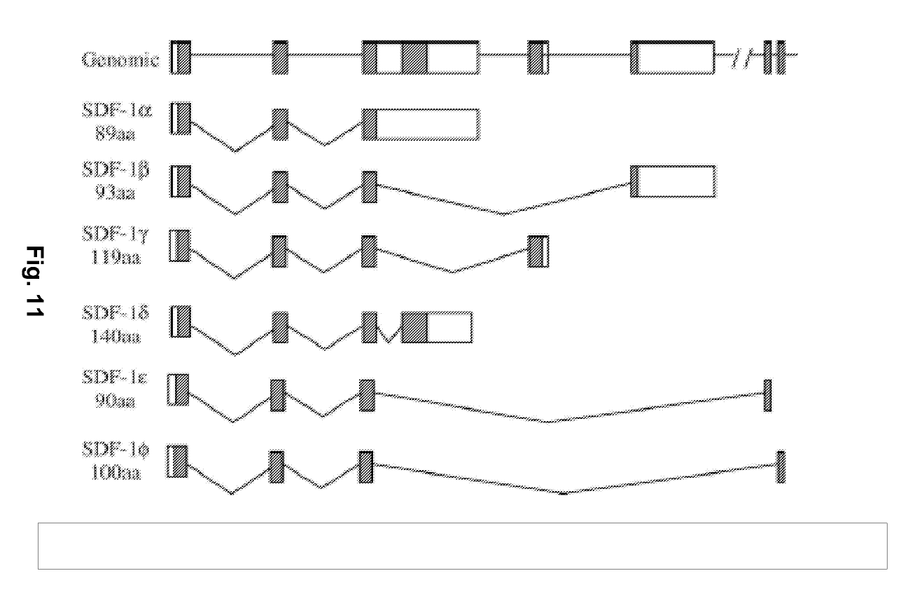

In sequence databases, splice variants not described in Ensembl were discovered: SDF-1 gamma, SDF-1 delta, SDF-1 epsilon and SDF-1 phi. All splice variants have the same first 3 exons. The last exon of SDF-1 epsilon and SDF-1 phi are located 72 kb downstream (see FIG. 11). These new sequences have been submitted to the NCBI in june 2006 by “Lilly Research Laboratories, Cardiovascular Division, Cancer Division and Integrative Biology, Eli Lilly and Company, Indianapolis, Ind. 46285, USA”. The cDNA (DQ345520 and DQ345519) encoding these two isoforms contain canonical splice sites, a polyadenylation signal and a polyA tail (not found on the genomic sequence).

Because all splice variants have the same first three exons, the 6 isoforms have the same N-ter part (88 amino acids).

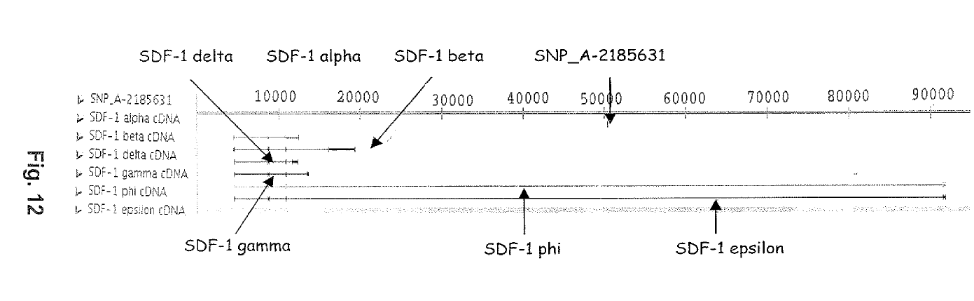

Thus, a detailed bio-analysis of the region around SNP_A-2185631 showed that:
the SDF-1 gene is longer than expected: 87 kb instead of 8 kb
the SNP of interest (SNP_A-2185631) is in the SDF-1 gene, located in the last intron of SDF-1 epsilon and SDF-1 phi (see FIG. 12).

It is thus concluded that the SDF-1 gene is associated with primary progressive MS.

### REFERENCES

1. Andriambeloson, E., Baillet, C., Vitte, P. A., Garotta, G., Dreano, M., Callizot, N. (2006) Interleukin-6 attenuates the development of experimental diabetes-related neuropathy. Neuropathology. 26, 32-42.
2. Altschul, S. F., Gish, W., Miller, W., Myers, E. W., and Lipman, D. J. (1990). Basic local alignment search tool. J. Mol. Biol. 215, 403-410.
3. Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
4. Amara, A., Lorthioir, O., Valenzuela, A., Magerus, A., Thelen, M., Montes, M., Virelizier, J. L., Delepierre, M., Baleux, F., Lortat-Jacob, H., and Arenzana-Seisdedos, F. (1999). Stromal cell-derived factor-1 alpha associates with heparan sulfates through the first beta-strand of the chemokine. J Biol Chem 274, 23916-23925.
5. Ambrosini, E., Remoli, M. E., Giacomini, E., Rosicarelli, B., Serafini, B., Lande, R., Aloisi, F., and Coccia, E. M. (2005). Astrocytes produce dendritic cell-attracting chemokines in vitro and in multiple sclerosis lesions. J Neuropathol. Exp Neurol. 64, 706-715.
6. Bajetto, A., Bonavia, R., Barbero, S., Florio, T., and Schettini, G. (2001). Chemokines and their receptors in the central nervous system. Front Neuroendocrinol. 22, 147-184.
7. Bezzi, P., Domercq, M., Brambilla, L., Galli, R., Schols, D., De Clercq, E., Vescovi, A., Bagetta, G., Kollias, G., Meldolesi, J., and Volterra, A. (2001). CXCR4-activated astrocyte glutamate release via TNFalpha: amplification by microglia triggers neurotoxicity. Nat Neurosci 4, 702-710.
8. Bianchi, R., Buyukakilli, B., Brines, M., Savino, C., Cavaletti, G., Oggioni, N., Lauria, G., Borgna, M., Lombardi, R., Cimen, B., Comelekoglu, U., Kanik, A., Tataroglu, C., Cerami, A., Ghezzi, P. (2004) Erythropoietin both protects from and reverses experimental diabetic neuropathy. Proc Natl Acad Sci USA. 101, 823-828.
9. Bottenstein, J. E. and Sato, G. H. (1979). Growth of a rat neuroblastoma cell line in serum-free supplemented medium. Proc Natl Acad Sci USA 76, 514-517.
10. Calcutt, N. A., Jorge, M. C., Yaksh T. L., Chaplan, S. R. (1996) Tactile allodynia and formalin hyperalgesia in streptozotocin-diabetic rats: effects of insulin, aldose reductase inhibition and lidocaine Pain 68, 293-9.
11. Calcutt N. A., Li L, Yaksh, T. L., Malmberg, A. B. (1995) Different effects of two aldose reductase inhibitors on nociception and prostaglandin E. Eur. J. Pharmacol. 285 189-197.
12. Cameron N. E., Cotter M. A., Low P. A. (1991) Nerve blood flow in early experimental diabetes in rats: relation to conduction deficits. Am. J. Physiol 261, E1-8.
13. Consilvio, C., Vincent, A. M., and Feldman, E. L. (2004). Neuroinflammation, COX-2, and ALS—a dual role? Exp Neurol. 187, 1-10.
14. Devereux, J., Haeberli, P., and Smithies, O. (1984). A comprehensive set of sequence analysis programs for the VAX. Nucleic Acids Res. 12, 387-395.
15. Dyck, P. J. B., Dyck, P, J. (1999) Diabetic polyneuropathy. In Diabetic Neuropathy, Dyck P J, Thomas P K (eds). Saunders W B: Philadelphia, Pa., 255-278.
16. Eikelenboom, P., Bate, C., Van Gool, W. A., Hoozemans, J. J., Rozemuller, J. M., Veerhuis, R., and Williams, A. (2002). Neuroinflammation in Alzheimer's disease and prion disease. Glia 40, 232-239.
17. Gao, H. M., Liu, B., Zhang, W., and Hong, J. S. (2003). Novel anti-inflammatory therapy for Parkinson's disease. Trends Pharmacol Sci 24, 395-401.
18. Gleichmann, M., Gillen, C., Czardybon, M., Bosse, F., Greiner-Petter, R., Auer, J., and Muller, H. W. (2000). Cloning and characterization of SDF-1gamma, a novel SDF-1 chemokine transcript with developmentally regulated expression in the nervous system. Eur J Neurosci 12, 1857-1866.
19. Grantham, R. (1974). Amino acid difference formula to help explain protein evolution. Science 185, 862-864.
20. Hesselgesser, J., Taub, D., Baskar, P., Greenberg, M., Hoxie, J., Kolson, D. L., and Horuk, R. (1998). Neuronal apoptosis induced by HIV-1 gp120 and the chemokine SDF-1 alpha is mediated by the chemokine receptor CXCR4. Curr Biol 8, 595-598.
21. Hoogewerf, A. J., Kuschert, G. S., Proudfoot, A. E., Borlat, F., Clark-Lewis, I., Power, C. A., and Wells, T. N. (1997). Glycosaminoglycans mediate cell surface oligomerization of chemokines. Biochemistry 36, 13570-13578.
22. Hunter, C. L., Bachman, D., and Granholm, A. C. (2004). Minocycline prevents cholinergic loss in a mouse model of Down's syndrome. Ann. Neurol. 56, 675-688.
23. Infante, J., Llorca, J., Berciano, J., and Combarros, O. (2005). Interleukin-8, intercellular adhesion molecule-1 and tumour necrosis factor-alpha gene polymorphisms and the risk for multiple system atrophy. J. Neurol. Sci 228, 11-13.
24. Jakobsen, J. (1976) Axonal dwindling in early experimental diabetes. II. A study of isolated nerve fibres. Diabetologia 12, 547-553.
25. Jiang, Y., Salafranca, M. N., Adhikari, S., Xia, Y., Feng, L., Sonntag, M. K., deFiebre, C. M., Pennell, N. A., Streit, W. J., and Harrison, J. K. (1998). Chemokine receptor expression in cultured glia and rat experimental allergic encephalomyelitis. J. Neuroimmunol. 86, 1-12.
26. Lazarini, F., Tham, T. N., Casanova, P., Arenzana-Seisdedos, F., and Dubois-Dalcq, M. (2003). Role of the alpha-chemokine stromal cell-derived factor (SDF-1) in the developing and mature central nervous system. Glia 42, 139-148.
27. Lehnardt, S., Lachance, C., Patrizi, S., Lefebvre, S., Follett, P. L., Jensen, F. E., Rosenberg, P. A., Volpe, J. J., and Vartanian, T. (2002). The toll-like receptor TLR4 is necessary for lipopolysaccharide-induced oligodendrocyte injury in the CNS. J Neurosci 22, 2478-2486.
28. Lehnardt, S., Massillon, L., Follett, P., Jensen, F. E., Ratan, R., Rosenberg, P. A., Volpe, J. J., and Vartanian, T. (2003). Activation of innate immunity in the CNS triggers neurodegeneration through a Toll-like receptor 4-dependent pathway. Proc Natl Acad Sci USA 100, 8514-8519.
29. Ma, Q., Jones, D., Borghesani, P. R., Segal, R. A., Nagasawa, T., Kishimoto, T., Bronson, R. T., and Springer, T. A. (1998). Impaired B-lymphopoiesis, myelopoiesis, and derailed cerebellar neuron migration in C. Proc Natl Acad Sci USA 95, 9448-9453.
30. Noseworthy, J. H. (1999). Progress in determining the causes and treatment of multiple sclerosis. Nature 399, A40-A47.
31. Panenka, W., Jijon, H., Herx, L. M., Armstrong, J. N., Feighan, D., Wei, T., Yong, V. W., Ransohoff, R. M., and MacVicar, B. A. (2001). P2X7-like receptor activation in astrocytes increases chemokine monocyte chemoattractant protein-1 expression via mitogen-activated protein kinase. J Neurosci 21, 7135-7142.
32. Pearson, W. R. (1990). Rapid and sensitive sequence comparison with FASTP and FASTA. Methods Enzymol. 183, 63-98.
33. Pearson, W. R. and Lipman, D. J. (1988). Improved tools for biological sequence comparison. Proc. Natl. Acad. Sci. U.S. A 85, 2444-2448.
34. Perry, V. H. (2004). The influence of systemic inflammation on inflammation in the brain: implications for chronic neurodegenerative disease. Brain Behav. Immun. 18, 407-413.
35. Phizicky, E. M. and Fields, S. (1995). Protein-protein interactions: methods for detection and analysis. Microbiol. Rev 59, 94-123.
36. Robinson, S., Tani, M., Strieter, R. M., Ransohoff, R. M., and Miller, R. H. (1998). The chemokine growth-regulated oncogene-alpha promotes spinal cord oligodendrocyte precursor proliferation. J Neurosci 18, 10457-10463.
37. Rovaris, M., Judica, E., Gallo, A., Benedettim B., Sormani, M. P., Caputo, D., Ghezzi, A., Montanari, E., Bertoolotto, A., Mancardi, G., Bergamaschi, R., Mrtinelli, V., Comi, G., Filippi, M. (2006) Grey matter damage predicts the evolution of primary progressive multiple sclerosis at 5 years. Brain, August 18, 1-7.
38. Sadir, R., Baleux, F., Grosdidier, A., Imberty, A., and Lortat-Jacob, H. (2001). Characterization of the stromal cell-derived factor-1alpha-heparin complex. J Biol Chem 276, 8288-8296.
39. Sahrbacher, U. C., Lechner, F., Eugster, H. P., Frei, K., Lassmann, H., and Fontana, A. (1998). Mice with an inactivation of the inducible nitric oxide synthase gene are susceptible to experimental autoimmune encephalomyelitis. Eur J Immunol 28, 1332-1338.
40. Schwarz, M. K. and Wells, T. N. (1999). Interfering with chemokine networks—the hope for new therapeutics. Curr Opin Chem Biol 3, 407-417.
41. Sima, A. A., Nathaniel, V., Bril V., McEwen T. A., Greene D. A. (1988) Histopathological heterogeneity of neuropathy in insulin-dependent and non-insulin-dependent diabetes, and demonstration of axo-glial dysjunction in human diabetic neuropathy. J. Clin. Invest 81, 349-364.
42. Sima A. A. (1994) Pathological definition and evaluation of diabetic neuropathy and clinical correlations. Can. J. Neurol. Sci. 21,S13-17.
43. Smith, T. F. and Waterman, M. S. (1981). Identification of common molecular subsequences. J. Mol. Biol. 147, 195-197.
44. Sorensen, T. L., Trebst, C., Kivisakk, P., Klaege, K. L., Majmudar, A., Ravid, R., Lassmann, H., Olsen, D. B., Strieter, R. M., Ransohoff, R. M., and Sellebjerg, F. (2002). Multiple sclerosis: a study of CXCL10 and CXCR3 co-localization in the inflamed central nervous system. J Neuroimmunol. 127, 59-68.
45. Stoll, G., Jander, S., and Myers, R. R. (2002). Degeneration and regeneration of the peripheral nervous system: from Augustus Waller's observations to neuroinflammation. J Peripher. Nerv. Syst. 7, 13-27.
46. Stumm, R. K., Rummel, J., Junker, V., Culmsee, C., Pfeiffer, M., Krieglstein, J., Hollt, V., and Schulz, S. (2002). A dual role for the SDF-1/CXCR4 chemokine receptor system in adult brain: isoform-selective regulation of SDF-1 expression modulates CXCR4-dependent neuronal plasticity and cerebral leukocyte recruitment after focal ischemia. J Neurosci 22, 5865-5878.
47. Tuppo, E. E. and Arias, H. R. (2005). The role of inflammation in Alzheimer's disease. Int. J Biochem Cell Biol 37, 289-305.
48. Yu, L. et al (2006), Identification and expression of novel isoforms of human stromal cell-derived factor 1.
49. Zhu, Y., Yu, T., Zhang, X. C., Nagasawa, T., Wu, J. Y., and Rao, Y. (2002). Role of the chemokine SDF-1 as the meningeal attractant for embryonic cerebellar neurons. Nat Neurosci 5, 719-720.
50. Zou, Y. R., Kottmann, A. H., Kuroda, M., Taniuchi, I., and Littman, D. R. (1998). Function of the chemokine receptor CXCR4 in haematopoiesis and in cerebellar development. Nature 393, 595-599.
51. Zujovic, V., Benavides, J., Vige, X., Carter, C., and Taupin, V. (2000). Fractalkine modulates TNF-alpha secretion and neurotoxicity induced by microglial activation. Glia 29, 305-315.

1-28. (canceled)

29. A method of treating a neurological disease comprising the administration of a pharmaceutical composition comprising SDF-1 or naturally occurring isoforms thereof to an individual in an amount sufficient to treat said neurological disease, wherein said neurological disease is selected from the group consisting of traumatic nerve injury, stroke, peripheral neuropathy, diabetic neuropathy, neuropathic pain, multiple sclerosis (MS), primary progressive multiple sclerosis (MS), secondary progressive multiple sclerosis (MS), chronic inflammatory multiple sclerosis, demyelinating polyneuropathy (CIDP) or Guillain-Barré syndrome (GBS).

30. The method of claim 29, wherein SDF-1 is selected from the group consisting of:
(a) a polypeptide comprising amino acids of SEQ ID NO: 1;
(b) a polypeptide comprising amino acids of SEQ ID NO: 4;
(c) a polypeptide comprising amino acids of SEQ ID NO: 7; and
(d) a polypeptide of (a) to (c) further comprising a signal sequence.

31. The method of claim 29, wherein SDF-1 is fused to a carrier molecule, a peptide or a protein that promotes the crossing of the blood brain barrier.

32. The method of claim 29, wherein SDF-1 is PEGylated.

33. The method of claim 29, wherein the fused protein comprises an immunoglobulin (Ig) fusion.

34. The method of claim 29, wherein said composition further comprises an interferon and/or osteopontin and/or clusterin.

35. The method of claim 34, wherein the interferon is interferon-β.

36. The method of claim 29, wherein SDF-1 is administered in an amount of about 0.001 to 1 mg/kg of body weight, or about 0.01 to 10 mg/kg of bodyweight or about 9, 8, 7, 6, 5, 4, 3, 2 or 1 mg/kg of body weight or about 0.1 to 1 mg/kg of body weight to said individual.
